(19) United States
(12) Patent Application Publication
(10) Pub. No.: US 2025/0297269 A1
(43) Pub. Date: Sep. 25, 2025

(54) INDUCIBLE PROMOTER FOR GENE EXPRESSION CONTROL

(71) Applicant: UT-Battelle, LLC, Oak Ridge, TN (US)

(72) Inventors: Carrie Eckert, Knoxville, TN (US); Nandhini Ashok, Oak Ridge, TN (US); Adam M. Guss, Oak Ridge, TN (US); Yasemin Kaygusuz, Knoxville, TN (US)

(21) Appl. No.: 19/088,072

(22) Filed: Mar. 24, 2025

Related U.S. Application Data
(60) Provisional application No. 63/569,340, filed on Mar. 25, 2024.

(51) Int. Cl. C12N 15/74; C12P 21/00

(52) U.S. Cl. CPC C12N 15/74; C12P 21/00; C12N 2800/101; C12N 2830/002

(57) ABSTRACT

The current disclosure relates to the present disclosure is directed to an inducible promoter system for use in a thermophilic bacterial cell. In another aspect, the disclosure is directed to a thermophilic host cell comprising the inducible promoter system described herein. In a further aspect, the disclosure is directed to a method of controlling gene expression in a thermophilic bacterium.

### CROSS REFERENCE TO RELATED APPLICATIONS

This application claims the benefit of U.S. Provisional Patent Application No. 63/569,340, filed Mar. 25, 2024, the contents of which is incorporated herein by reference in its entirety.

### STATEMENT REGARDING FEDERALLY SPONSORED RESEARCH

The United States Government has rights in this invention pursuant to contract no. DE-AC05-00OR22725 between the United States Department of Energy and UT-Battelle, LLC.

### INCORPORATION BY REFERENCE OF SEQUENCE LISTING

The Sequence Listing in an XML file, named as 44057_5602_1_SequenceListing 57,344 bytes, created on Mar. 17, 2025, and submitted to the United States Patent and Trademark Office via Patent Center, is incorporated herein by reference.

### BACKGROUND

Clostridium thermocellum (also known as Acetivibrio thermocellus,

Ruminiclostridium thermocellum, and Hungateiclostridium thermocellum) is an anaerobic thermophilic bacterium capable of metabolizing complex and heterogenous lignocellulosic biomass. C. thermocellum both deconstructs lignocellulose to soluble sugars and ferments the resulting sugars into commodity chemicals such as ethanol, acetate, lactate, formate, H2, isobutanol, 2,3 butanediol, and free amino acids. Research in the past decade has focused on metabolic engineering of C. thermocellum to make industrially significant amounts of a specific fuel or commodity chemical, primarily ethanol. The ability of C. thermocellum to complete the two step deconstruction and fermentation process eliminates an extra step of chemical processing of lignocellulosic biomass, in turn reducing the cost and complexity, in a process called consolidated bioprocessing (CBP). Improving the efficiency and yield of CBP performed by C. thermocellum requires identifying and characterizing new gene functions and strain engineering. However, the genetic engineering tools currently available to study and engineer C. thermocellum are limited due to its thermophilic growth conditions (50-60° C.), temperatures at which many standard genetic engineering tools are not functional.

One important class of genetic engineering tools that enables temporal and dosage control of gene expression is the inducible promoter. Previously, numerous inducible promoters have been developed for mesophilic Clostridium species. However, only one inducible promoter has been demonstrated in C. thermocellum. This inducible promoter is a native promoter found in the C. thermocellum ATCC 27405 genome, which is activated by adding laminaribiose into the culture media. The laminaribiose-inducible promoter is not ideal for C. thermocellum both because the native promoter in the genome could be affected by the laminaribiose induction and because laminaribiose is metabolized by C. thermocellum, which leads to unstable gene expression and reduced specificity. Also, laminaribiose is prohibitively expensive for large-scale experiments. In addition to inducible promoters, an alternative method of regulating gene expression is using a riboswitch, such as the pbuE riboswitch from Bacillus subtilis adapted for use in C. thermocellum. While the end goal is the same, the mechanism and details of our approach are completely different from the previously reported studies.

Currently, there are no sufficiently and tightly controlled inducible promoters with stable expression developed for C. thermocellum.

### SUMMARY

In one aspect, the present disclosure is directed to an inducible promoter system for use in a thermophilic bacterial cell. In another aspect, the disclosure is directed to a thermophilic host cell comprising the inducible promoter system described herein. In a further aspect, the disclosure is directed to a method of controlling gene expression in a thermophilic bacterium.

In one aspect, the present disclosure is directed to an inducible promoter system for use in a thermophilic bacterial cell. In some embodiments, the inducible promoter system comprises a combination of a first expression cassette and a second expression cassette, wherein the first expression cassette comprises a heterologous promoter operably linked to a nucleic acid encoding a regulatory protein, wherein the regulatory protein is a xylose regulator and a homolog of xylR; and wherein the second expression cassette comprises an inducible promoter operably linked to a heterologous nucleic acid sequence for a gene of interest, wherein activity of the inducible promoter is modulated in a thermophile by the regulatory protein in the first expression cassette.

In some embodiments the inducible promoter comprises a nucleic acid sequence as set forth in SEQ ID NO: 2. In some embodiments, the inducible promoter comprises a nucleic acid sequence comprising at least 80% sequence identity to SEQ ID NO: 1. In some embodiments, the inducible promoter comprises a nucleic acid sequence as set forth in SEQ ID NO: 1.

In some embodiments, the regulatory protein is a xylose regulator from a Caldicellulosiruptor bacterium. In some embodiments, the regulatory protein is encoded by a xylR from a Caldicellulosiruptor bacterium. In some embodiments, the Caldicellulosiruptor bacterium is a C. bescii, C. saccharolyticus, C. obsidiansis, C. hydrothermalis, C. owensensis, C. acetigenus, C. kronotskyensis, C. acetigenus, C. changbaiensis, C. naganoensis, C. morganii, C. danielii, or C. diazotrophicus bacterium. In some embodiments, the regulatory protein comprises an amino acid sequence comprising at least 90% sequence identity to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28. In some embodiments, the regulatory protein comprises an amino acid sequence as set forth in SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

In some embodiments, the two expression cassettes are placed on the same vector.

In some embodiments, the gene of interest is thermophilic Cas9 (GeoCas9), thermophilic Cas13 (TccCas13a), or encodes the protein of a thermostable beta-glucuronidase (gus-tr3337), the red fluorescent protein mScarlet3, the superfolder green fluorescent protein (sfGFP), pyruvate decarboxylase, or alcohol dehydrogenase.

In some embodiments, the thermophile is Clostridium thermocellum, Parageobacillus thermoglucosidasius (previously called Geobacillus thermoglucosidasius), or Bacillus licheniformis.

One aspect of the current disclosure is directed to a host cell comprising the combination of a first and a second expression cassettes wherein the first expression cassette comprises a heterologous promoter operably linked to a nucleic acid encoding a regulatory protein, wherein the regulatory protein is a xylose regulator and a homolog of xylR; and wherein the second expression cassette comprises an inducible promoter operably linked to a heterologous nucleic acid sequence for a gene of interest, wherein activity of the inducible promoter is modulated in a thermophile by the regulatory protein in the first expression cassette, and wherein the host cell is a thermophilic bacterium. In some embodiments, the inducible promoter comprises a nucleic acid sequence as set forth in SEQ ID NO: 2. In some embodiments, the inducible promoter comprises a nucleic acid sequence with 80% sequence identity to SEQ ID NO: 1. In some embodiments, the inducible promoter comprises a nucleic acid sequence as set forth in SEQ ID NO: 1.

In some embodiments, the regulatory protein is a xylose regulator from a Caldicellulosiruptor bacterium. In some embodiments, the regulatory protein is encoded by a xylR from a Caldicellulosiruptor bacterium. In some embodiments, the Caldicellulosiruptoris a C. bescii, C. saccharolyticus, C. obsidiansis, C. hydrothermalis, C. owensensis, C. acetigenus, C. kronotskyensis, C. acetigenus, C. changbaiensis, C. naganoensis, C. morganii, C. danielii, or C. diazotrophicus bacterium. In some embodiments, the regulatory protein comprises an amino acid sequence comprising at least 90% sequence identity to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28. In some embodiments, the regulatory protein comprises an amino acid sequence as set forth in SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

In some embodiments, the gene of interest is thermophilic Cas9 (GeoCas9), thermophilic Cas13 (TccCas13a) or encodes the protein of a thermostable beta-glucuronidase (gus-tr3337), the red fluorescent protein mScarlet3, the superfolder green fluorescent protein (sfGFP), pyruvate decarboxylase, or alcohol dehydrogenase.

In some embodiments, the second expression cassette is integrated into the genome of the host cell.

In some embodiments, the host cell is a Clostridium thermocellum, Parageobacillus thermoglucosidasius (previously called Geobacillus thermoglucosidasius), or Bacillus licheniformis.

One aspect of the current disclosure is directed to a method of controlling gene expression in a thermophilic bacterium, the method comprising:
(a) obtaining a host cell comprising the combination of a first and a second expression cassettes wherein the first expression cassette comprises a heterologous promoter operably linked to a nucleic acid encoding a regulatory protein, wherein the regulatory protein is a xylose regulator and a homolog of xylR; and wherein the second expression cassette comprises an inducible promoter operably linked to a heterologous nucleic acid sequence for a gene of interest, wherein activity of the inducible promoter is modulated in a thermophile by the regulatory protein in the first expression cassette, wherein the host cell is a thermophilic bacterium, and
(b) incubating the host cell in the presence of xylose under conditions that result in the expression of the gene of interest.

In some embodiments, the inducible promoter comprises a nucleic acid sequence as set forth in SEQ ID NO: 2. In some embodiments, the inducible promoter comprises a nucleic acid sequence comprising at least 80% sequence identity to SEQ ID NO: 1. In some embodiments, the inducible promoter comprises a nucleic acid sequence as set forth in SEQ ID NO: 1.

In some embodiments, the regulatory protein is a xylose regulator from a Caldicellulosiruptor bacterium. In some embodiments, the regulatory protein is encoded by a xylR from a Caldicellulosiruptor bacterium. In some embodiments, the Caldicellulosiruptor bacterium is a C. bescii, C. saccharolyticus, C. obsidiansis, C. hydrothermalis, C. owensensis, C. acetigenus, C. kronotskyensis, C. acetigenus, C. changbaiensis, C. naganoensis, C. morganii, C. danielii, or C. diazotrophicus bacterium. In some embodiments, the regulatory protein comprises an amino acid sequence comprising at least 90% sequence identity to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28. In some embodiments, the regulatory protein comprises an amino acid sequence as set forth in SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

In some embodiments, the two expression cassettes are placed on the same vector.

In some embodiments, the gene of interest is thermophilic Cas9 (GeoCas9), thermophilic Cas13 (TccCas13a), or encodes the protein of a thermostable beta-glucuronidase (gus-tr3337), the red fluorescent protein mScarlet3, the superfolder green fluorescent protein (sfGFP), pyruvate decarboxylase, or alcohol dehydrogenase.

In some embodiments, the thermophile is Clostridium thermocellum, Parageobacillus thermoglucosidasius (previously called Geobacillus thermoglucosidasius), or Bacillus licheniformis.

### BRIEF DESCRIPTION OF DRAWINGS

FIG. 1. Diagram representing the design of the genetic cassette constructed to regulate the superfolder green fluorescent protein (sfGFP) gene expression by the xylose-inducible promoter (Pate_0063, Caldicellulosiruptor bescii (ate)). The xylose-responsive transcription factor gene (xylR, Athe_0617, C. bescii) expression is driven by the Pctx_1194 promoter (Clostridium thermocellum (ctx)). T: Terminator.

FIG. 2. The regulatory response of the xylose-inducible promoter using sfGFP as a reporter in Clostridium thermocellum. The fluorescence units were measured with excitation at 488 nm and emission at 510 nm and were normalized to the optical density measured at 600 nm (OD600).

FIGS. 3A-B. FIG. 3A represents a multiple alignment of 200 bp upstream region of the xylose isomerase genes Athe_0603 in Caldicellulosiruptor bescii (SEQ ID NO: 8), Calkr_1977 in C. acetigenus I77R1B (SEQ ID NO: 10), SOJ16_000578 in C. danielii (SEQ ID NO: 11), Calhy_2055 in C. hydrothermalis (SEQ ID NO: 12), Calkro_2027 in C. kronotskyensis (SEQ ID NO: 13), COB47_0553 in C. obsidiansis (SEQ ID NO: 14), and Calow_0468 in C. owensensis (SEQ ID NO: 15) (analysis of the promoter region). Conserved xylose regulator (xylR) binding sites are indicated in a box. Sequence alignment was performed by Clustal W2. FIG. 3B represents a phylogenetic tree of the putative xylose regulator ROK family proteins Athe_0617 in Caldicellulosiruptor bescii, Csac_0695 in C. saccharolyticus, COB47_0572 in C. obsidiansis, Calhy_2039 in C. hydrothermalis, Calow_0493 in C. owensensis, Calkr_0571 in C. acetigenus I77R1B, Calkro_2009 in C. kronotskyensis, Calla_1782 in C. acetigenus 6A, ELD05_11440 in C. changbaiensis, OTJ99_000465 in C. naganoensis, OTK00_002089 in C. morganii, SOJ16_000597 in C. danielii, and the xylose regulator CaldiYA01_18370 in C. diazotrophicus constructed by neighbor-joining method. Sequence alignment was performed by ClustalW2 and phylogenetic tree was constructed by Simple Phylogeny.

FIG. 4. Diagram representing the design of the genetic cassette constructed to regulate the superfolder green fluorescent protein (sfGFP) gene expression by the xylose-inducible promoter (Pcdh_2055, Caldicellulosiruptor hydrothermalis (chd)). The xylose-responsive transcription factor gene (xylR, Athe_0617, C. bescii) expression is driven by the Pctx_1194 promoter (Clostridium thermocellum (ctx)). T: Terminator.

FIG. 5. The regulatory response of the xylose-inducible promoter (Pcdh_2055) using sfGFP as a reporter in Clostridium thermocellum. The fluorescence units were measured with excitation at 488 nm and emission at 510 nm and were normalized to the optical density measured at 600 nm (OD600).

FIG. 6A-B. A) shows the regulatory response of the xylose-inducible promoter (Pate_0603) using sfGFP as a reporter in Bacillus licheniformis at 37° C. B) shows the regulatory response of the xylose-inducible promoter (Pate_0603) using sfGFP as a reporter in Bacillus licheniformis at 50° C. For each, fluorescence units were measured with excitation at 488 nm and emission at 510 nm and were normalized to the optical density measured at 600 nm (OD600).

### DETAILED DESCRIPTION

In one aspect, the present disclosure is directed to an inducible promoter system for use in a thermophilic bacterial cell. In another aspect, the disclosure is directed to a thermophilic host cell comprising the inducible promoter system described herein. In a further aspect, the disclosure is directed to a method of controlling gene expression in a thermophilic bacterium.

Unless otherwise noted, technical terms are used according to conventional usage. Definitions of common terms in molecular biology can be found in Benjamin Lewin, Genes VII, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

As used herein, the singular forms “a,” “an,” and “the,” refer to both the singular as well as plural, unless the context clearly indicates otherwise. As used herein, the term “comprises” means “includes.” Thus, “comprising a nucleic acid molecule” means “including a nucleic acid molecule” without excluding other elements. It is further to be understood that any and all base sizes given for nucleic acids are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. All references, including patent applications and patents, are herein incorporated by reference in their entireties.

As used herein, “bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

The terms “genetically modified,” “genetically engineered,” “recombinant cell,” and “recombinant strain” are used interchangeably herein and refer to bacterial cells that have been genetically modified by the cloning and transformation methods of the present disclosure. Thus, the terms include a prokaryote that has been genetically altered, modified, or engineered, such that it exhibits an altered, modified, or different genotype and/or phenotype (e.g., when the genetic modification affects coding nucleic acid sequences of the microorganism), as compared to the naturally occurring microorganism from which it was derived. It is understood that the terms refer not only to the particular recombinant microorganism in question, but also to the progeny or potential progeny of such a microorganism. Non-viral vectors and transfection reagents, including cationic lipids or non-liposomal lipids, are non-limiting examples of safe tools for introducing exogenous nucleic acids into cells. Transfected nucleic acids are incorporated into the endosome via endocytosis and exposed to the cytosol upon endosome rupture. In some instances, a cell is genetically altered through introducing exogenous nucleic acid without integrating into its genome. This modification is a temporary modification known as a transient transfection, which will not be included in progeny. An example of such genetic alteration occurs with plasmids. Plasmids offer a flexible and reversible way to modify gene expression without making permanent changes to the host genome. Expression levels can be tuned by controlling plasmid copy number or through the activity of plasmid-encoded regulators.

As used herein, the term “nucleic acid” refers to a polymeric form of nucleotides of any length, either ribonucleotides or deoxyribonucleotides, or analogs thereof. This term refers to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like.

As used herein, the term “gene” refers to any segment of DNA associated with a biological function. Thus, genes include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” is known in the art and refers to related sequences that share a common ancestor or family member and are determined based on the degree of sequence identity. The terms “homology,” “homologous,” “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure, homologous sequences are compared. “Homologous sequences”, “homologs”, or “orthologs” are thought, believed, or known to be functionally related. A functional relationship may be indicated in any one of a number of ways, including, but not limited to: (a) degree of sequence identity and/or (b) the same or similar biological function.

Preferably, both (a) and (b) are indicated. Homology can be determined using software programs readily available in the art, such as those discussed in Current Protocols in Molecular Biology (F. M. Ausubel et al., eds., 1987) Supplement 30, section 7.718, Table 7.71. Examples of alignment programs include but are not limited to: Mac Vector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, “promoter” refers to a DNA sequence capable of controlling the expression of a coding sequence or functional RNA. The promoter sequence may consist of proximal and more distal upstream elements, the latter elements often referred to as enhancers.

As used herein, the term “heterologous” refers to a relationship or linkage of two nucleic acid or protein sequences that is not naturally found in a particular organism.

As used herein, the term “exogenous” refers to a substance coming from a source other than its native source. For example, the terms “exogenous protein” and “exogenous gene” refer to a protein and gene that have been artificially supplied to a biological system (e.g., tissue, cell or intracellular site) from a source non-native to the biological system. Artificially mutated variants of endogenous genes are considered “exogenous” for the purposes of this disclosure.

The term “operably linked” means in this context the sequential arrangement of the promoter polynucleotide according to the disclosure with a further oligo- or polynucleotide, resulting in transcription of the further polynucleotide. In some embodiments, the promoter sequences of the present disclosure are inserted just prior to a gene's 5′UTR, or open reading frame. In other embodiments, the operably linked promoter sequences and gene sequences of the present disclosure are separated by one or more linker nucleotides.

Serine recombinase Assisted Genome Engineering (SAGE) is a method that has been recently developed for rapid and simple genomic integration of genetic cassettes into model and non-model organisms like Escherichia coli, Pseudomonas sp., Rhodococcus jostii, and

Rhodopseudomonas palustris. SAGE uses a large serine recombinase to facilitate a site-specific recombination event between two non-identical base pair DNA sequences, called attB and attP sites. Recombination between these attB and attP sites, collectively called att sites, results in the formation of new attL and attR sites, leaving genetic “scars” that are not substrates for further recombination, making the recombination reaction irreversible and stable. This is unlike the FLP-frt and the CRE-lox tyrosine recombinase-mediated systems, which are reversible and can result in strain instability issues when used for genome engineering. SAGE has been shown to work in mesophilic organisms, but not yet in thermophilic organisms.

As used herein, a “system” refers to a combination of multiple components or products which can interact in a way to produce a desired result. In some embodiments, the components could be provided in the form of a kit. In some embodiments, the disclosure provides kits containing any one or more of the elements disclosed in the above methods and compositions. In some embodiments, the kit comprises a vector system and instructions for using the kit.

### Inducible Promoter System

In one aspect, the present disclosure is directed to an inducible promoter system for use in a thermophilic bacterial cell. In some embodiments, the inducible promoter system comprises a combination of a first expression cassette and a second expression cassette, wherein the first expression cassette comprises a heterologous promoter operably linked to a nucleic acid encoding a regulatory protein, wherein the regulatory protein is a xylose regulator and a homolog of xylR; and wherein the second expression cassette comprises an inducible promoter operably linked to a heterologous nucleic acid sequence for a gene of interest, wherein activity of the inducible promoter is modulated in a thermophile by the regulatory protein in the first expression cassette.

As used herein, the term “expression cassette” refers to a DNA comprising a gene and a regulatory sequence (such as a transcriptionally regulatory sequence, e.g., a promoter). In a successful transformation of the expression cassette into a cell, the expression cassette directs the cellular machinery to make RNA and/or protein.

In some embodiments, the inducible promoter comprises a nucleic acid sequence comprising at least 80% sequence identity to SEQ ID NO: 1. In some embodiments, the inducible promoter comprises a nucleic acid sequence comprising at least 85% sequence identity to SEQ ID NO: 1. In some embodiments, the inducible promoter comprises a nucleic acid sequence comprising at least 90% sequence identity to SEQ ID NO: 1. In some embodiments, the inducible promoter comprises a nucleic acid sequence comprising at least 91% sequence identity to SEQ ID NO: 1. In some embodiments, the inducible promoter comprises a nucleic acid sequence comprising at least 92% sequence identity to SEQ ID NO: 1. In some embodiments, the inducible promoter comprises a nucleic acid sequence comprising at least 93% sequence identity to SEQ ID NO: 1. In some embodiments, the inducible promoter comprises a nucleic acid sequence comprising at least 94% sequence identity to SEQ ID NO: 1. In some embodiments, the inducible promoter comprises a nucleic acid sequence comprising at least 95% sequence identity to SEQ ID NO: 1. In some embodiments, the inducible promoter comprises a nucleic acid sequence comprising at least 96% sequence identity to SEQ ID NO: 1. In some embodiments, the inducible promoter comprises a nucleic acid sequence comprising at least 97% sequence identity to SEQ ID NO: 1. In some embodiments, the inducible promoter comprises a nucleic acid sequence comprising at least 98% sequence identity to SEQ ID NO: 1. In some embodiments, the inducible promoter comprises a nucleic acid sequence comprising at least 99% sequence identity to SEQ ID NO: 1. In some embodiments, the inducible promoter comprises a nucleic acid sequence as set forth in SEQ ID NO: 1.

SEQ ID NO: 1
TCATGCATATTCTGGAAAAGAAAGCATGTCGGAAATCACCATAGCTATA
CATCCCAGCACCACAGCTCTGTCTTTCAGTTTTGAAACCTCTATCTTAA
GATTATAAAACTGGGCGATAAAGGATCTCTGGTTAATTACTTCCCTCAA
TTTTTCTAAAAACAACTCTCCAAAAAATGATGCCTTATTACCTATAATT
ACCATTTCAGGATTAAAAATATTTACAAGATTTGCCACGCCTATCCCCA
TCTTTTCAGCAACCTCAAGTATAGCCATTCTGCAAACTCTGCTTCCTTC
TTTTGCTGCTTGTATTATCCGAGAAGGAGTGATTTCATCTACATTTTCC
CAGCTTATATACCTATCCTCTACTCCTTGTTTTACAAGCTTTTTTATAA
CACTCAAAAGTGCCCTCTCGGATGCAAAGTTCTCAAGACAGCCAATATT
GCCGCAGCTGCAAACATCGTCCTGAAAGTTGATAGTGGTATGTCCAACT
TCACCTGCAAATCCTGCAGCACCTCTGAAAAGTTTGTTGTCGATAATAA
TTCCTGCACCAAGCCCAATTCCAACACTCAAATAAATCAAATCACTAAC
TTTTCCCCACTCACCAAACCACTTTTCGCCCAGTGCGCCTGCATTTGCT
TCATTGTCAATATAAACAGGGAGGTTGAACTTTTGCTGAACAATTGACC
TCAAAGGGACATTTTGCCATTTCAAATTGGGAGCAAGAAGGACAGTTCC
AGACTCTTTTTCTATAATACCTGGAACACCAATTCCAATACCTAAAATC
CCTTTTGGAGTTTGTGGCGCCTTTTTTACTGATTTTTCAATCAGGTCAA
AAAGAAGCCTTAAAAGTTTTTCCTTATCCTCACCTATTTTCATGTTGGC
ATATTCTTCAAAAATAACTTCTCCCACAAAGTTTGAGAGAATAATATGA
ATATAGTCAACACCTAAGTCAATTCCGATGATTGAACCTACATCTTTGT
TTACCTGTAAAAGGACAGGTCTTCTTCCGCCTTTGGACTTACCGTATCC
TTTTTCTACTACATACCCTTCTTTTATGAGCTCATCGGTGAGGTTTGAG
ACTGTTGCTTTATTTAAATCTACAAGTTTCGATATCTTTGTACGAGATA
TTATCTTGTTGTCCAAAATTGTTTTCAAAACCAAAAGCTTGTTTATTTG
CTTTAGTAGCGTGTGGTTACCCATAG

In some embodiments the inducible promoter comprises a core nucleic acid sequence. The core nucleic acid sequence comprises the xylose regulator xylR binding site. In some embodiments the inducible promoter comprises a nucleic acid sequence as set forth in SEQ ID NO: 2.

SEQ ID NO: 2 GTTTGTTTAATAAACAAACTAAG

In some embodiments, the inducible promoter comprises the core nucleic acid sequence and functions to activate the transcription of the target gene. Non-limiting examples of such embodiments include, SEQ ID NO: 8, SEQ ID NO: 10, SEQ ID NO: 11, SEQ ID NO: 12, SEQ ID NO: 13, SEQ ID NO: 14, and SEQ ID NO: 15.

In some embodiments, the inducible promoter comprises the core nucleic acid sequence and comprises at least 80% (i.e., at least 85%, at least 90%, at least 91%, at least 92%, at least 93%, at least 94%, at least 95%, at least 96%, at least 97%, at least 98%, and at least 99%) sequence identity to a nucleic acid as laid out in SEQ ID NO: 8, SEQ ID NO: 10, SEQ ID NO: 11, SEQ ID NO: 12, SEQ ID NO: 13, SEQ ID NO: 14, or SEQ ID NO: 15.

In some embodiments, the inducible promoter is between 180 and 220 base pairs in length. In some embodiments, the inducible promoter is between 185 and 215 base pairs in length. In some embodiments, the inducible promoter is between 190 and 210 base pairs in length. In some embodiments, the inducible promoter is between 195 and 205 base pairs in length.

In some embodiments, the inducible promoter comprises the core nucleic acid and is between 180 and 220 base pairs in length.

In some embodiments, the inducible promoter comprises the core nucleic acid, is between 180 and 220 base pairs in length, and comprises at least 80% (i.e., at least 85%, at least 90%, at least 91%, at least 92%, at least 93%, at least 94%, at least 95%, at least 96%, at least 97%, at least 98%, and at least 99%) sequence identity to a nucleic acid as laid out in SEQ ID NO: 8, SEQ ID NO: 10, SEQ ID NO: 11, SEQ ID NO: 12, SEQ ID NO: 13, SEQ ID NO: 14, or SEQ ID NO: 15

In some embodiments, the regulatory protein is a xylose regulator from a Caldicellulosiruptor bacterium. In some embodiments, the regulatory protein is encoded by a xylR from a Caldicellulosiruptor bacterium. In some embodiments, the Caldicellulosiruptor bacterium is a C. bescii, C. saccharolyticus, C. obsidiansis, C. hydrothermalis, C. owensensis, C. acetigenus, C. kronotskyensis, C. acetigenus, C. changbaiensis, C. naganoensis, C. morganii, C. danielii, or C. diazotrophicus bacterium.

In some embodiments, the regulatory protein comprises an amino acid sequence comprising at least 90% sequence identity to SEQ ID NO: 16. In some embodiments, the regulatory protein comprises an amino acid sequence comprising at least 91% sequence identity to SEQ ID NO: 16. In some embodiments, the regulatory protein comprises an amino acid sequence comprising at least 92% sequence identity to SEQ ID NO: 16. In some embodiments, the regulatory protein comprises an amino acid sequence comprising at least 93% sequence identity to SEQ ID NO: 16. In some embodiments, the regulatory protein comprises an amino acid sequence comprising at least 94% sequence identity to SEQ ID NO: 16. In some embodiments, the regulatory protein comprises an amino acid sequence comprising at least 95% sequence identity to SEQ ID NO: 16. In some embodiments, the regulatory protein comprises an amino acid sequence comprising at least 96% sequence identity to SEQ ID NO: 16. In some embodiments, the regulatory protein comprises an amino acid sequence comprising at least 97% sequence identity to SEQ ID NO: 16. In some embodiments, the regulatory protein comprises an amino acid sequence comprising at least 98% sequence identity to SEQ ID NO: 16. In some embodiments, the regulatory protein comprises an amino acid sequence comprising at least 99% sequence identity to SEQ ID NO: 16. In some embodiments, the regulatory protein comprises an amino acid sequence as set forth in SEQ ID NO: 16.

In some embodiments, the regulatory protein comprises an amino acid sequence comprising at least 90% sequence identity to SEQ ID NO: 17. In some embodiments, the regulatory protein comprises an amino acid sequence comprising at least 91% sequence identity to SEQ ID NO: 17. In some embodiments, the regulatory protein comprises an amino acid sequence comprising at least 92% sequence identity to SEQ ID NO: 17. In some embodiments, the regulatory protein comprises an amino acid sequence comprising at least 93% sequence identity to SEQ ID NO: 17. In some embodiments, the regulatory protein comprises an amino acid sequence comprising at least 94% sequence identity to SEQ ID NO: 17. In some embodiments, the regulatory protein comprises an amino acid sequence comprising at least 95% sequence identity to SEQ ID NO: 17. In some embodiments, the regulatory protein comprises an amino acid sequence comprising at least 96% sequence identity to SEQ ID NO: 17. In some embodiments, the regulatory protein comprises an amino acid sequence comprising at least 97% sequence identity to SEQ ID NO: 17. In some embodiments, the regulatory protein comprises an amino acid sequence comprising at least 98% sequence identity to SEQ ID NO: 17. In some embodiments, the regulatory protein comprises an amino acid sequence comprising at least 99% sequence identity to SEQ ID NO: 17. In some embodiments, the regulatory protein comprises an amino acid sequence as set forth in SEQ ID NO: 17.

In some embodiments, the regulatory protein comprises an amino acid sequence comprising at least 90% sequence identity to SEQ ID NO: 18. In some embodiments, the regulatory protein comprises an amino acid sequence comprising at least 91% sequence identity to SEQ ID NO: 18. In some embodiments, the regulatory protein comprises an amino acid sequence comprising at least 92% sequence identity to SEQ ID NO: 18. In some embodiments, the regulatory protein comprises an amino acid sequence comprising at least 93% sequence identity to SEQ ID NO: 18. In some embodiments, the regulatory protein NO: 18. In some embodiments, the regulatory protein comprises an amino acid sequence comprising at least 95% sequence identity to SEQ ID NO: 18. In some embodiments, the regulatory protein comprises an amino acid sequence comprising at least 96% sequence identity to SEQ ID NO: 18. In some embodiments, the regulatory protein comprises an amino acid sequence comprising at least 97% sequence identity to SEQ ID NO: 18. In some embodiments, the regulatory protein comprises an amino acid sequence comprising at least 98% sequence identity to SEQ ID NO: 18. In some embodiments, the regulatory protein comprises an amino acid sequence comprising at least 99% sequence identity to SEQ ID NO: 18. In some embodiments, the regulatory protein comprises an amino acid sequence as set forth in SEQ ID NO: 18.

In some embodiments, the regulatory protein comprises an amino acid sequence comprising at least 90% sequence identity to SEQ ID NO: 19. In some embodiments, the regulatory protein comprises an amino acid sequence comprising at least 91% sequence identity to SEQ ID NO: 19. In some embodiments, the regulatory protein comprises an amino acid sequence comprising at least 92% sequence identity to SEQ ID NO: 19. In some embodiments, the regulatory protein comprises an amino acid sequence comprising at least 93% sequence identity to SEQ ID NO: 19. In some embodiments, the regulatory protein comprises an amino acid sequence comprising at least 94% sequence identity to SEQ ID NO: 19. In some embodiments, the regulatory protein comprises an amino acid sequence comprising at least 95% sequence identity to SEQ ID NO: 19. In some embodiments, the regulatory protein comprises an amino acid sequence comprising at least 96% sequence identity to SEQ ID NO: 19. In some embodiments, the regulatory protein comprises an amino acid sequence comprising at least at least 97% sequence identity to SEQ ID NO: 19. In some embodiments, the regulatory protein comprises an amino acid sequence comprising at least at least 98% sequence identity to SEQ ID NO: 19. In some embodiments, the regulatory protein comprises an amino acid sequence comprising at least at least 99% sequence identity to SEQ ID NO: 19. In some embodiments, the regulatory protein comprises an amino acid sequence as set forth in SEQ ID NO: 19.

In some embodiments, the regulatory protein comprises an amino acid sequence comprising at least at least 90% sequence identity to SEQ ID NO: 20. In some embodiments, the regulatory protein comprises an amino acid sequence comprising at least at least 91% sequence identity to SEQ ID NO: 20. In some embodiments, the regulatory protein comprises an amino acid sequence comprising at least 92% sequence identity to SEQ ID NO: 20. In some embodiments, the regulatory protein comprises an amino acid sequence comprising at least 93% sequence identity to SEQ ID NO: 20. In some embodiments, the regulatory protein comprises an amino acid sequence comprising at least 94% sequence identity to SEQ ID NO: 20. In some embodiments, the regulatory protein comprises an amino acid sequence comprising at least 95% sequence identity to SEQ ID NO: 20. In some embodiments, the regulatory protein comprises an amino acid sequence comprising at least 96% sequence identity to SEQ ID NO: 20. In some embodiments, the regulatory protein comprises an amino acid sequence comprising at least 97% sequence identity to SEQ ID NO: 20. In some embodiments, the regulatory protein comprises an amino acid sequence comprising at least 98% sequence identity to SEQ ID NO: 20. In some embodiments, the regulatory protein comprises an amino acid sequence comprising at least 99% sequence identity to SEQ ID NO: 20. In some embodiments, the regulatory protein comprises an amino acid sequence as set forth in SEQ ID NO: 20.

In some embodiments, the regulatory protein comprises an amino acid sequence comprising at least 90% sequence identity to SEQ ID NO: 21. In some embodiments, the regulatory protein comprises an amino acid sequence comprising at least 91% sequence identity to SEQ ID NO: 21. In some embodiments, the regulatory protein comprises an amino acid sequence comprising at least 92% sequence identity to SEQ ID NO: 21. In some embodiments, the regulatory protein comprises an amino acid sequence comprising at least 93% sequence identity to SEQ ID NO: 21. In some embodiments, the regulatory protein comprises an amino acid sequence comprising at least 94% sequence identity to SEQ ID NO: 21. In some embodiments, the regulatory protein comprises an amino acid sequence comprising at least 95% sequence identity to SEQ ID NO: 21. In some embodiments, the regulatory protein comprises an amino acid sequence comprising at least 96% sequence identity to SEQ ID NO: 21. In some embodiments, the regulatory protein comprises an amino acid sequence comprising at least 97% sequence identity to SEQ ID NO: 21. In some embodiments, the regulatory protein comprises an amino acid sequence comprising at least 98% sequence identity to SEQ ID NO: 21. In some embodiments, the regulatory protein comprises an amino acid sequence comprising at least 99% sequence identity to SEQ ID NO: 21. In some embodiments, the regulatory protein comprises an amino acid sequence as set forth in SEQ ID NO: 21.

In some embodiments, the regulatory protein comprises an amino acid sequence comprising at least 90% sequence identity to SEQ ID NO: 22. In some embodiments, the regulatory protein comprises an amino acid sequence comprising at least 91% sequence identity to SEQ ID NO: 22. In some embodiments, the regulatory protein comprises an amino acid sequence comprising at least 92% sequence identity to SEQ ID NO: 22. In some embodiments, the regulatory protein comprises an amino acid sequence comprising at least 93% sequence identity to SEQ ID NO: 22. In some embodiments, the regulatory protein comprises an amino acid sequence comprising at least 94% sequence identity to SEQ ID NO: 22. In some embodiments, the regulatory protein comprises an amino acid sequence comprising at least 95% sequence identity to SEQ ID NO: 22. In some embodiments, the regulatory protein comprises an amino acid sequence comprising at least 96% sequence identity to SEQ ID NO: 22. In some embodiments, the regulatory protein comprises an amino acid sequence comprising at least 97% sequence identity to SEQ ID NO: 22. In some embodiments, the regulatory protein comprises an amino acid sequence comprising at least 98% sequence identity to SEQ ID NO: 22. In some embodiments, the regulatory protein comprises an amino acid sequence comprising at least 99% sequence identity to SEQ ID NO: 22. In some embodiments, the regulatory protein comprises an amino acid sequence as set forth in SEQ ID NO: 22.

In some embodiments, the regulatory protein comprises an amino acid sequence comprising at least 90% sequence identity to SEQ ID NO: 23. In some embodiments, the regulatory protein comprises an amino acid sequence comprising at least 91% sequence identity to SEQ ID NO: 23. In some embodiments, the regulatory protein comprises an amino acid sequence comprising at least 92% sequence identity to SEQ ID NO: 23. In some embodiments, the regulatory protein comprises an amino acid sequence comprising at least 93% sequence identity to SEQ ID NO: 23. In some embodiments, the regulatory protein NO: 23. In some embodiments, the regulatory protein comprises an amino acid sequence comprising at least 95% sequence identity to SEQ ID NO: 23. In some embodiments, the regulatory protein comprises an amino acid sequence comprising at least 96% sequence identity to SEQ ID NO: 23. In some embodiments, the regulatory protein comprises an amino acid sequence comprising at least comprising at least 97% sequence identity to SEQ ID NO: 23. In some embodiments, the regulatory protein comprises an amino acid sequence comprising at least comprising at least 98% sequence identity to SEQ ID NO: 23. In some embodiments, the regulatory protein comprises an amino acid sequence comprising at least comprising at least 99% sequence identity to SEQ ID NO: 23. In some embodiments, the regulatory protein comprises an amino acid sequence as set forth in SEQ ID NO: 23.

In some embodiments, the regulatory protein comprises an amino acid sequence comprising at least 90% sequence identity to SEQ ID NO: 24. In some embodiments, the regulatory protein comprises an amino acid sequence comprising at least 91% sequence identity to SEQ ID NO: 24. In some embodiments, the regulatory protein comprises an amino acid sequence comprising at least 92% sequence identity to SEQ ID NO: 24. In some embodiments, the regulatory protein comprises an amino acid sequence comprising at least 93% sequence identity to SEQ ID NO: 24. In some embodiments, the regulatory protein comprises an amino acid sequence comprising at least 94% sequence identity to SEQ ID NO: 24. In some embodiments, the regulatory protein comprises an amino acid sequence comprising at least 95% sequence identity to SEQ ID NO: 24. In some embodiments, the regulatory protein comprises an amino acid sequence comprising at least 96% sequence identity to SEQ ID NO: 24. In some embodiments, the regulatory protein comprises an amino acid sequence comprising at least 97% sequence identity to SEQ ID NO: 24. In some embodiments, the regulatory protein comprises an amino acid sequence comprising at least 98% sequence identity to SEQ ID NO: 24. In some embodiments, the regulatory protein comprises an amino acid sequence comprising at least 99% sequence identity to SEQ ID NO: 24. In some embodiments, the regulatory protein comprises an amino acid sequence as set forth in SEQ ID NO: 24.

In some embodiments, the regulatory protein comprises an amino acid sequence comprising at least 90% sequence identity to SEQ ID NO: 25. In some embodiments, the regulatory protein comprises an amino acid sequence comprising at least 91% sequence identity to SEQ ID NO: 25. In some embodiments, the regulatory protein comprises an amino acid sequence comprising at least 92% sequence identity to SEQ ID NO: 25. In some embodiments, the regulatory protein comprises an amino acid sequence comprising at least 93% sequence identity to SEQ ID NO: 25. In some embodiments, the regulatory protein comprises an amino acid sequence comprising at least 94% sequence identity to SEQ ID NO: 25. In some embodiments, the regulatory protein comprises an amino acid sequence comprising at least 95% sequence identity to SEQ ID NO: 25. In some embodiments, the regulatory protein comprises an amino acid sequence comprising at least 96% sequence identity to SEQ ID NO: 25. In some embodiments, the regulatory protein comprises an amino acid sequence comprising at least 97% sequence identity to SEQ ID NO: 25. In some embodiments, the regulatory protein comprises an amino acid sequence comprising at least 98% sequence identity to SEQ ID NO: 25. In some embodiments, the regulatory protein comprises an amino acid sequence comprising at least 99% sequence identity to SEQ ID NO: 25. In some embodiments, the regulatory protein comprises an amino acid sequence as set forth in SEQ ID NO: 25.

In some embodiments, the regulatory protein comprises an amino acid sequence comprising at least 90% sequence identity to SEQ ID NO: 26. In some embodiments, the regulatory protein comprises an amino acid sequence comprising at least 91% sequence identity to SEQ ID NO: 26. In some embodiments, the regulatory protein comprises an amino acid sequence comprising at least 92% sequence identity to SEQ ID NO: 26. In some embodiments, the regulatory protein comprises an amino acid sequence comprising at least 93% sequence identity to SEQ ID NO: 26. In some embodiments, the regulatory protein comprises an amino acid sequence comprising at least 94% sequence identity to SEQ ID NO: 26. In some embodiments, the regulatory protein comprises an amino acid sequence comprising at least 95% sequence identity to SEQ ID NO: 26. In some embodiments, the regulatory protein comprises an amino acid sequence comprising at least 96% sequence identity to SEQ ID NO: 26. In some embodiments, the regulatory protein comprises an amino acid sequence comprising at least 97% sequence identity to SEQ ID NO: 26. In some embodiments, the regulatory protein comprises an amino acid sequence comprising at least 98% sequence identity to SEQ ID NO: 26. In some embodiments, the regulatory protein comprises an amino acid sequence comprising at least 99% sequence identity to SEQ ID NO: 26. In some embodiments, the regulatory protein comprises an amino acid sequence as set forth in SEQ ID NO: 26.

In some embodiments, the regulatory protein comprises an amino acid sequence comprising at least 90% sequence identity to SEQ ID NO: 27. In some embodiments, the regulatory protein comprises an amino acid sequence comprising at least 91% sequence identity to SEQ ID NO: 27. In some embodiments, the regulatory protein comprises an amino acid sequence comprising at least 92% sequence identity to SEQ ID NO: 27. In some embodiments, the regulatory protein comprises an amino acid sequence comprising at least 93% sequence identity to SEQ ID NO: 27. In some embodiments, the regulatory protein comprises an amino acid sequence comprising at least 94% sequence identity to SEQ ID NO: 27. In some embodiments, the regulatory protein comprises an amino acid sequence comprising at least 95% sequence identity to SEQ ID NO: 27. In some embodiments, the regulatory protein comprises an amino acid sequence comprising at least 96% sequence identity to SEQ ID NO: 27. In some embodiments, the regulatory protein comprises an amino acid sequence comprising at least 97% sequence identity to SEQ ID NO: 27. In some embodiments, the regulatory protein comprises an amino acid sequence comprising at least 98% sequence identity to SEQ ID NO: 27. In some embodiments, the regulatory protein comprises an amino acid sequence comprising at least 99% sequence identity to SEQ ID NO: 27. In some embodiments, the regulatory protein comprises an amino acid sequence as set forth in SEQ ID NO: 27.

In some embodiments, the regulatory protein comprises an amino acid sequence comprising at least 90% sequence identity to SEQ ID NO: 28. In some embodiments, the regulatory protein comprises an amino acid sequence comprising at least 91% sequence identity to SEQ ID NO: 28. In some embodiments, the regulatory protein comprises an amino acid sequence comprising at least 92% sequence identity to SEQ ID NO: 28. In some embodiments, the regulatory protein comprises an amino acid sequence comprising at least 93% sequence identity to SEQ ID NO: 28. In some embodiments, the regulatory protein NO: 28. In some embodiments, the regulatory protein comprises an amino acid sequence comprising at least 95% sequence identity to SEQ ID NO: 28. In some embodiments, the regulatory protein comprises an amino acid sequence comprising at least 96% sequence identity to SEQ ID NO: 28. In some embodiments, the regulatory protein comprises an amino acid sequence comprising at least 97% sequence identity to SEQ ID NO: 28. In some embodiments, the regulatory protein comprises an amino acid sequence comprising at least 98% sequence identity to SEQ ID NO: 28. In some embodiments, the regulatory protein comprises an amino acid sequence comprising at least 99% sequence identity to SEQ ID NO: 28. In some embodiments, the regulatory protein comprises an amino acid sequence as set forth in SEQ ID NO: 28.

In some embodiments, the two expression cassettes are placed on the same vector, i.e. cassette 1 and cassette 2 are on the same vector.

In some embodiments, the gene of interest is thermophilic Cas9 (GeoCas9). In some embodiments, the gene of interest is thermophilic Cas13 (TccCas13a). In some embodiments, the gene of interest encodes the protein of a thermostable beta-glucuronidase (gus-tr3337). In some embodiments, the gene of interest encodes the protein of a red fluorescent protein mScarlet3. In some embodiments, the gene of interest encodes the protein of a pyruvate decarboxylase. In some embodiments, the gene of interest encodes the protein of an alcohol dehydrogenase.

Thermophiles are heat-loving organisms that exhibit optimal growth at a temperature at or above 50° C. As such, thermophilic bacteria are bacteria which grow and thrive at temperatures of 50° C. or more. There are several known thermophilic bacteria known in the art. Thermophiles are inhabitants of various ecological niches like deep sea hydrothermal vents, terrestrial hot springs, and other extreme geographical/geological sites including volcanic sites, tectonically active faults as well as decaying matters such as the compost and deep organic landfills. Non-limiting examples of thermophilic bacteria include bacteria from the genus of: Bacillus, Geobacillus, Paenebacillus, Clostridium, Anaerocellum,
Caldicellulosiruptor, Thermus, Pyrococcus, Thermococcus, Thermoanaerobacter, Thermoplasma, Thermosipho, Thermoanaerobacterium, Herbinix, Acetivibrio, Acidothermus, Hydrogenobaculum, Rhodoplanes, Ornithinibacillus, Thermaerobacter, Fervidobacterium, and Persephonella, among others.

In some embodiments, the thermophilic bacterium cell is any bacterial cell that can grow and thrive at thermophilic temperatures. In some embodiments, the thermophile bacterium cell is from the genus of Bacillus, Geobacillus, Paenebacillus, Clostridium, Anaerocellum, Caldicellulosiruptor, Thermus, Pyrococcus, Thermococcus, Thermoanaerobacter, Thermoanaerobacterium, Herbinix, Acetivibrio, or Acidothermus. In some embodiments, the thermophile is Clostridium thermocellum, Geobacillus, or Bacillus licheniformis. In some embodiments, the bacterium cell is a Clostridium thermocellum, Parageobacillus thermoglucosidasius (previously called Geobacillus thermoglucosidasius), or Bacillus licheniformis.

### Methods of Thermostable Insertion

In some embodiments of the disclosure, the inducible promoter is inserted into a chromosome of a thermophilic bacterium. In some embodiments, the inducible promoter is incorporated into the genome of the thermophilic bacterium. In some embodiments, the inducible promoter is not incorporated into the genome, rather the promoter is present during regulated transcription.

The inducible promoter can be inserted into the thermophilic bacterium in a matter of ways known in the art. One thermostable method of insertion is the serine recombinase-assisted genome engineering (tSAGE) technique described in PCT/US2024/055875, which is herein incorporated by reference in its entirety. tSAGE comprises:
a) transfecting a cargo plasmid into a genetically engineered thermophile bacterial cell comprising at least one att site in its chromosome wherein the att site is one member of a pair of attB and attP recombination sites,
wherein the thermophile bacterial cell expresses a thermophilic site-specific recombinase that recognizes the pair of attB and attP recombination sites;
wherein the cargo plasmid comprising a corresponding att recombination site, wherein the corresponding att recombination site is the other member of the pair of attB and attP recombination sites;
wherein the cargo plasmid comprises the heterologous DNA;
wherein the cargo plasmid is capable of insertion into the chromosome of the thermophile bacterial cell at the at least one att site via recombination with the at least one corresponding att site on the cargo plasmid; and
wherein the recombination results in the formation of an attL site and an attR site,
wherein the attL and attR sites are not substrates for further recombination.
wherein the insertion is a site-specific recombination at a temperature at or above 50° C. and is thermostable; and
b) selecting a thermophile bacterial cell wherein the heterologous DNA is inserted into the chromosome of the thermophile bacterial cell.

In some embodiments, the expression of the thermophilic site-specific recombinase is achieved by transfecting, in step (a), a helper plasmid into the thermophile bacterial cell, wherein the helper plasmid comprises a nucleic acid sequence encoding the site-specific recombinase.

In some embodiments, the method further comprises a step c) culturing the selected bacterial cell under conditions suitable for growth and replication. In some embodiments, the culturing comprises culturing at a temperature at or above 55° C.

In some embodiments, the cargo plasmid further comprises a selectable marker gene. Selectable marker genes are genes that are added to cells to give them a trait that makes them easy to identify and select. Selectable marker genes are well-known in the art and include, but are not limited to, antibiotic resistance genes and visual reporter genes. In some embodiments, the selection of a thermophilic bacterial cell wherein the heterologous DNA is inserted into the chromosome of the thermophile bacterial cell of step (b) is based on the selectable marker.

In some embodiments, the selectable marker gene is flanked by another pair of attB and attP recombination sites recognized by another thermophilic site-specific recombinase. In some embodiments, the selectable marker gene is removed from the selected thermophile bacterial cell via recombination mediated by the another thermophilic site-specific recombinase. In some embodiments, the another thermophilic site-specific recombinase is expressed from a helper plasmid introduced into the selected thermophile bacterial cell. In some embodiments, the thermophilic site-specific recombinase and the another thermophilic site-specific recombinase are a serine recombinase or a tyrosine recombinase. For example, the att pair used for integration of the cargo plasmid into the bacterial chromosome is an attBlattP pair specific to a Y412MC61 recombinase, while another pair of att recombination sites is a pair of attBlattP recombination sites which flank the selectable marker gene that is located in the cargo DNA which gets inserted into the bacterial chromosome. The additional pair of att recombination sites is a pair of attBlattP recombination sites that are specific to the BXB1 recombinase. As such, the additional pair of att recombination sites flanking the selectable marker gene allows for the selectable marker gene to be excised from the bacterial chromosome through the use of BXB1 recombinase once the selectable marker is no longer needed. In some embodiments, the serine recombinase is Y412MC61, BXB1, or TG1.

In some embodiments of the method for the thermostable insertion of a heterologous DNA into a chromosome of an organism, the thermophile bacterial cell comprises multiple att sites in its chromosome, wherein the method comprises transfecting into the thermophile bacterial cell another cargo plasmid comprising another corresponding att site and another heterologous DNA, and selecting a thermophile bacterial cell in which the another heterologous DNA is also integrated in the chromosome. In some embodiments, the multiple att sites are used for serial insertion, i.e. multiple insertions that occur one after another. For example, the bacterial cell chromosome comprises multiple attB sites, each site being specific for a different recombinase. A cargo plasmid has a cognate attP site specific for the Y412MC61 recombinase, so the attB site in the bacterial chromosome specific for the Y412MC61 recombinase works with the Y412MC61 attP site on the cargo plasmid to integrate the cargo plasmid into the bacterial chromosome. Then, an additional cargo plasmid having a cognate attP site for the BXB1 recombinase so that the attB site in the bacterial chromosome specific for the BXB1 recombinase works with the cognate BXB1 att site in the additional cargo plasmid to insert the additional cargo plasmid into the bacterial chromosome.

### Methods of Controlling Gene Expression

One aspect of the current disclosure is directed to a method of controlling gene expression in a thermophilic bacterium, the method comprising:
(a) obtaining a host cell according to methods described herein, and
(b) incubating the host cell in the presence of xylose under conditions that result in the expression of the gene of interest.

In some embodiments, the inducible promoter comprises a nucleic acid sequence as set forth in SEQ ID NO: 2. In some embodiments, the inducible promoter comprises a nucleic acid sequence comprising at least 80% sequence identity to SEQ ID NO: 1. In some embodiments, the inducible promoter comprises a nucleic acid sequence as set forth in SEQ ID NO: 1.

In some embodiments, the regulatory protein is a xylose regulator from a Caldicellulosiruptor bacterium. In some embodiments, the regulatory protein is encoded by a xylR from a Caldicellulosiruptor bacterium. In some embodiments, the Caldicellulosiruptor bacterium is a C. bescii, C. saccharolyticus, C. obsidiansis, C. hydrothermalis, C. owensensis, C. acetigenus, C. kronotskyensis, C. acetigenus, C. changbaiensis, C. naganoensis, C. morganii, C. danielii, or C. diazotrophicus bacterium. In some embodiments, the regulatory protein comprises an amino acid sequence comprising at least 90% sequence identity to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28. In some embodiments, the regulatory protein comprises an amino acid sequence as set forth in SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

In some embodiments, the two expression cassettes are placed on the same vector.

In some embodiments, the gene of interest is thermophilic Cas9 (GeoCas9), thermophilic Cas13 (TccCas13a), or encodes the protein of a thermostable beta-glucuronidase (gus-tr3337), the red fluorescent protein mScarlet3, pyruvate decarboxylase, or alcohol dehydrogenase.

In some embodiments, the thermophilic bacterial cell is from the genus of Bacillus, Geobacillus, Paenebacillus, Clostridium, Anaerocellum, Caldicellulosiruptor, Thermus, Pyrococcus, Thermococcus, Thermoanaerobacter, Thermoanaerobacterium, Herbinix, Acetivibrio, or Acidothermus. In some embodiments, the thermophile is Clostridium thermocellum, Geobacillus, or Bacillus licheniformis. In some embodiments, the bacterium cell is a Clostridium thermocellum, Geobacillus thermoglucosidasius, or Bacillus licheniformis.

### Examples

The following examples are set forth as being representative of the present disclosure. These examples are not to be construed as limiting the scope of the present disclosure as these and other equivalent embodiments will be apparent in view of the present disclosure, figures and accompanying claims.

### Example 1: Design of heterologous inducible promoter for a thermobacterium

Inducible promoters and riboswitches are essential tools in synthetic biology that enable time—and dose-dependent gene expression. Therefore, chromosomally encoded, regulated gene expression systems were explored.

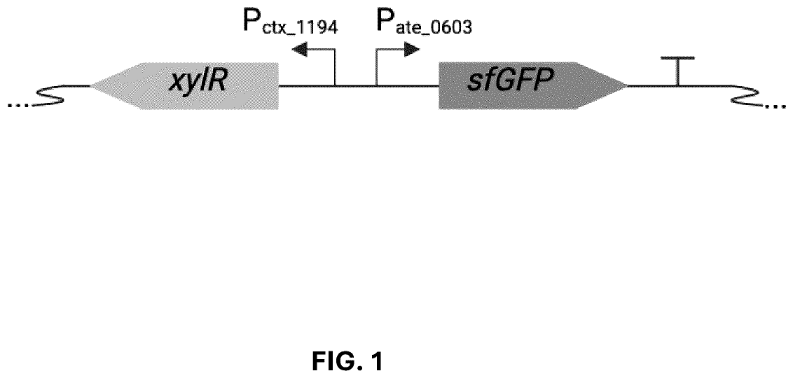

A heterologous inducible promoter was designed to achieve stable and sustained gene expression in C. thermocellum. Inducing the promoter was accomplished with xylose, an inexpensive, non-metabolizable compound. DNA sequences of the xylose-inducible promoter and the xylose regulator (xylR, Athe_0617) of C. bescii were identified from the previously published literature-inducible promoter system from C. bescii to design the inducible gene expression system. We constructed a gene expression cassette to express both the xylR gene driven by the Pctx_1194 promoter isolated from C. thermocellum and the superfolder green fluorescence protein (sfGFP) under the control of the xylose-inducible promoter (Pate_0063) identified in the C. bescii genome (FIG. 1). The Pctx_1194 promoter is native to C. thermocellum and was demonstrated to be a strong promoter in C. thermocellum.

The expression cassette was inserted into an integrating vector to be genomically integrated into the C. thermocellum AG8235 strain using the recently developed thermostable version of the serine recombinase-assisted genome engineering (SAGE) technique 16. AG8235 was derived from LL1299, a C. thermocellum strain evolved under laboratory conditions after deleting the restriction enzyme (Clo1313_0478) (Table 1). AG8235 carries a poly-attB landing pad, including the Y412MC61 attB site replacing the Clo1313_2366 locus in the genome (Table 1). The integrating vector carries a Y412MC61 integrase attP site, a colEl origin for replication in Escherichia coli, and a PGAPDH promoter-driven thiamphenicol resistance gene (Table 2). The genome integration of the expression cassette was performed through the serine recombinase-assisted genome engineering (tSAGE) technique as described in PCT/US2024/055875, which is incorporated herein by reference in its entirety. Briefly, tSAGE was achieved by co-transforming the AG8235 electrocompetent cells with both the integrating plasmid carrying the expression cassette and the plasmid that encodes the serine recombinase from Geobacillus sp., Y412MC61.

### Example 2: Confirmation of Xylose Inducible Promoter

After confirming the tSAGE-mediated genomic integration of the inducible gene expression cassette by polymerase chain reaction, the resulting strain was anaerobically grown at 50° C. in CTFUD medium supplemented with both 15 μg/ml thiamphenicol and D-(+)-xylose at given concentrations ranging from 0-50 mM for 48 hours. The cells were washed three times aerobically with phosphate-buffered saline (PBS) and resuspended in PBS for incubation overnight in the dark. The optical density (OD600)-normalized sfGFP fluorescence of the cultures was measured as a readout of the inducible promoter activity.

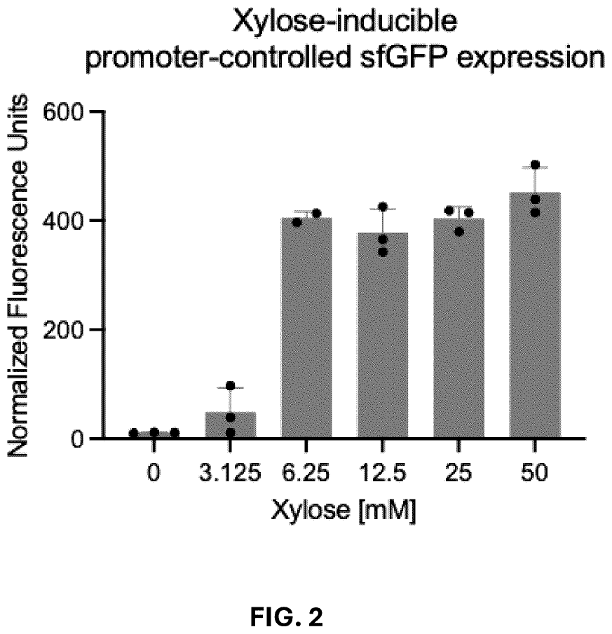

The xylose-inducible promoter from C. bescii showed approximately 40-fold induction in sfGFP expression when induced with xylose at 6.25 mM or higher concentrations (FIG. 2). In the absence of xylose, the expression level is low, indicating tight control of gene expression. Thus, we demonstrate that the xylose-inducible promoter from C. bescii with the corresponding XylR transcription factor is an ideal inducible promoter to achieve tight control over the time and dosage of gene expression in the thermophilic bacterium C. thermocellum.

The xylose-inducible system disclosed herein uses a low-cost inducer molecule not catabolized by C. thermocellum and does not control the expression of native C. thermocellum genes, making it a highly desirable expression system for C. thermocellum. Moreover, the xylose-inducible promoter can also be activated by the xylose generated by the degradation of lignocellulose during CBP to drive gene expression for the next steps, such as fermentation. The inducible promoter will help accelerate the identification of gene functions and strain engineering to improve the efficiency and yield of biofuel during CBP.

### Example 3. Identification of xylR homologs

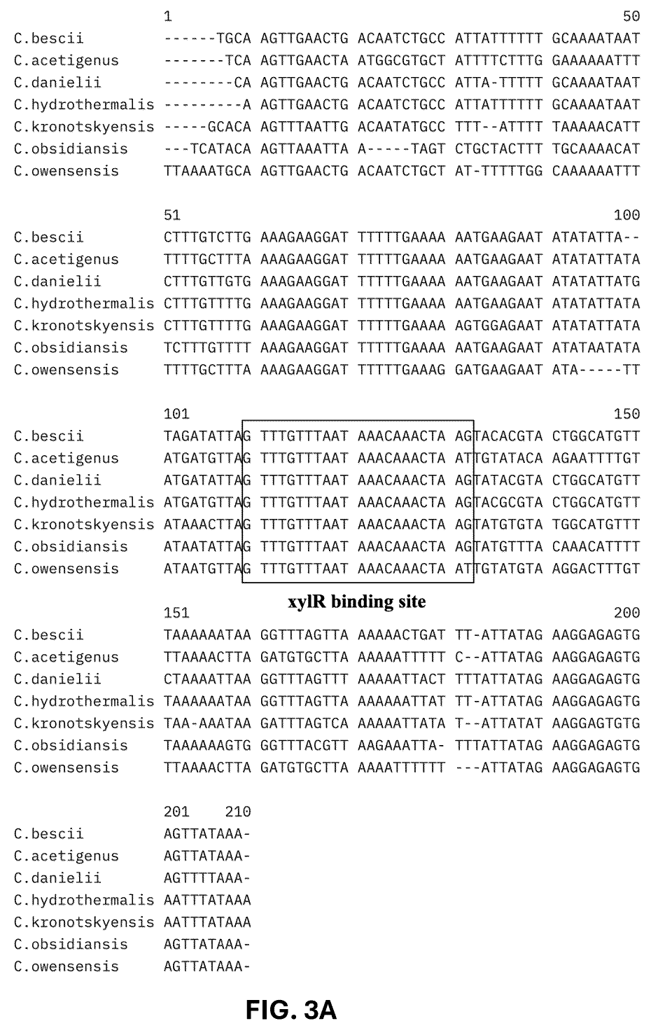

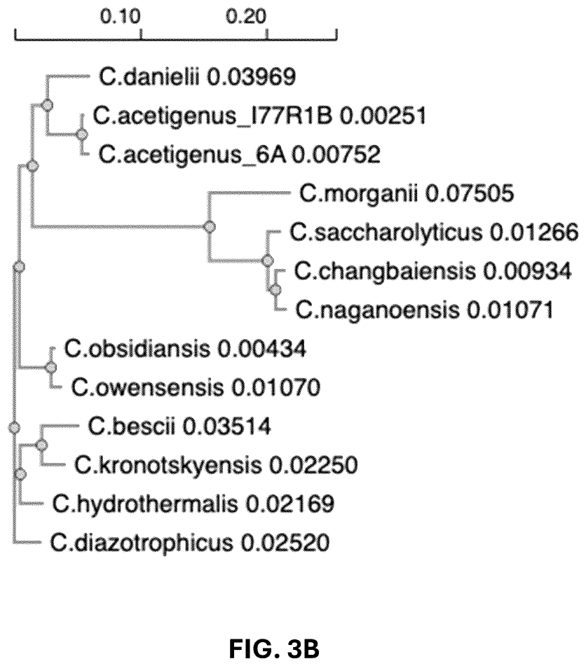

The sequence of the xylose isomerase genes in six different Caldicellulosiruptor species show high similarity to the sequence of the xylose isomerase gene of Caldicellulosiruptor bescii. The xylose regulator (XylR) binding site present in the C. bescii xylose isomerase gene promoter (SEQ ID NO: 2: GTTTGTTTAATAAACAAACTAAG) was found in the xylose isomerase gene promoters in all six species analyzed (FIG. 3A).

Homologs of the xylose regulator (xylR) were identified in twelve different Caldicellulosiruptor species through a multiple alignment of the putative xylose regulator ROK family proteins: Athe_0617 in Caldicellulosiruptor bescii, Csac_0695 in C. saccharolyticus, COB47_0572 in C. obsidiansis, Calhy_2039 in C. hydrothermalis, Calow_0493 in C. owensensis, Calkr_0571 in C. acetigenus I77R1B, Calkro_2009 in C. kronotskyensis, Calla_1782 in C. acetigenus 6A, ELD05_11440 in C. changbaiensis, OTJ99_000465 in C. naganoensis, OTK00_002089 in C. morganii, SOJ16_000597 in C. danielii, and the xylose regulator CaldiYA01_18370 in C. diazotrophicus. Additionally, a phylogenetic tree of the putative xylose regulator ROK family proteins was constructed by neighbor-joining method (FIG. 3B).

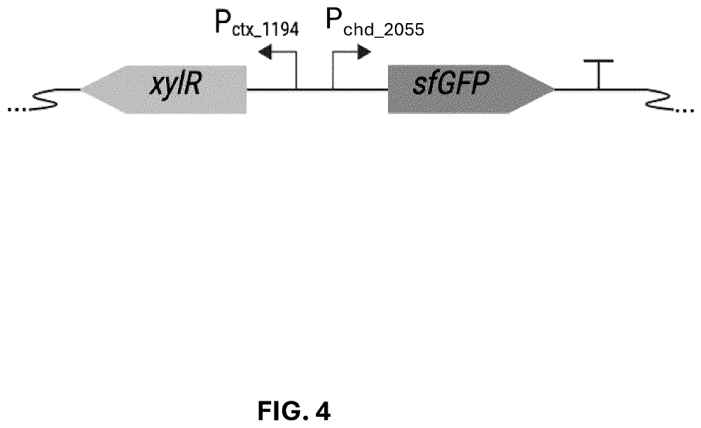

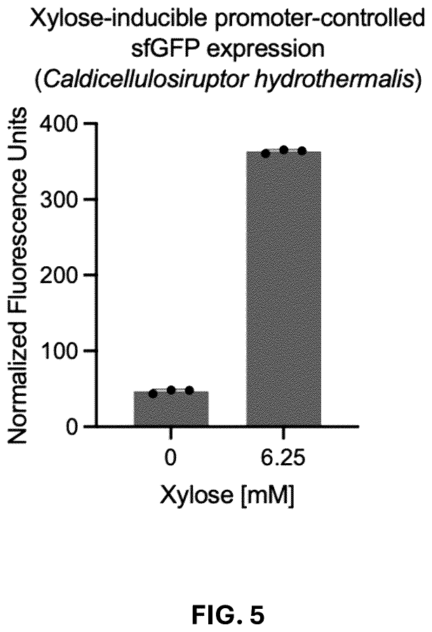

Xylose isomerase promoter homologs from six of these species were tested for their ability to drive xylose-inducible gene expression in Clostridium thermocellum. The regulatory response of the xylose-inducible promoter from Caldicellulosiruptor hydrothermalis (Pchd_2055) was tested in Clostridium thermocellum using the superfolder green fluorescence protein (sfGFP) as a reporter. To do so, a gene expression cassette was constructed to express both the xylR gene driven by the Pctx_1194 promoter isolated from C. thermocellum and the sfGFP under the control of the xylose-inducible promoter (Pchd_2055) identified in the C. hydrothermalis genome (FIG. 4). As described above, the Pctx_1194 promoter is native to C. thermocellum. The Pchd_2055 xylose-inducible promoter of C. hydrothermalis showed approximately 7-fold induction in sfGFP expression when induced with xylose at 6.25 mM (FIG. 5).

Xylose inducible promoter homologs from five additional Caldicellulosiruptor species were unable to be tested as there were issues integrating them into the C. thermocellum genome (data not shown). Additionally, the xylose-inducible promoter from Clostridioides difficile, the cumate inducible promoter from Pseudomonas putida, and the arabinose inducible promoter from E. coli were also tested; however, they were not inducible in C. thermocellum (data not shown).

As such, the xylose-inducible promoters from two separate Caldicellulosiruptor species, C. bescii and C. hydrothermalis, were successful in driving sfGFP in C. thermocellum.

### Example 4. Testing of Promoter in Other Thermophilic Bacteria

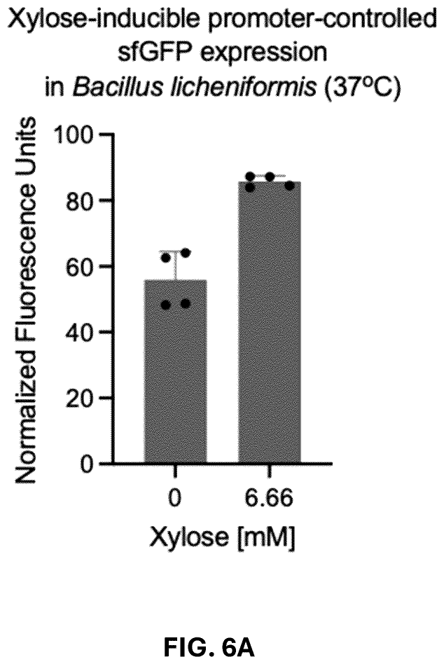

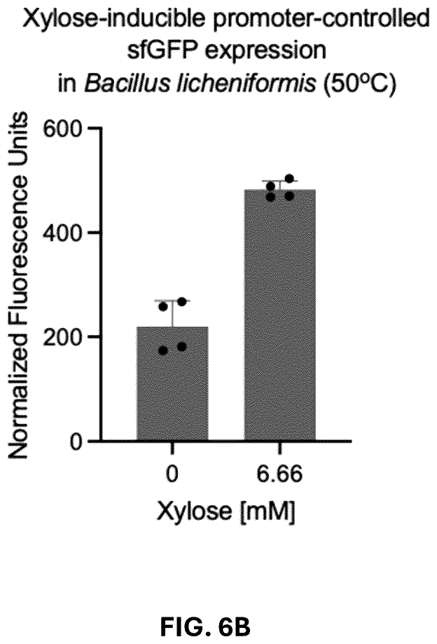

The C. bescii xylose inducible promoter was tested for functionality in a different thermophilic bacteria host, Bacillus licheniformis. The C. bescii xylose inducible promoter showed an approximately 1.5 fold induction in sfGFP expression when induced with xylose at a 6.66 mM concentration at 37° C. (FIG. 6A); additionally, the C. bescii xylose inducible promoter showed an approximately 2.5 fold induction in sfGFP expression when induced with xylose at a 6.66 mM concentration at 50° C. (FIG. 6B).

### Example 5: General materials and methods

Strains and culturing conditions

The parent C. thermocellum strain used in this work is LL1299, a strain evolved in laboratory conditions after deleting the Clo1313_0478 gene. AG8235 is derived from LL1299 by genomic insertion of a poly-attB landing pad sequence by homologous recombination replacing the Clo1313_2366 locus. The poly-attB landing pad includes the attB site of the integrase used in this work, Y412MC6118. AG8235 was thiamphenicol sensitive and was cultured in liquid CTFUD. Following the transformation with the plasmids, the strain with the genomically integrated expression cassette was plated on CTFUD-agar with 15 μg/ml thiamphenicol. Colonies from the agar plates were picked into liquid CTFUD with 15 μg/ml thiamphenicol. The strain was grown in liquid CTFUD with 15 μg/ml thiamphenicol and D-(+)-xylose at given concentrations for 48 hours for the sfGFP assay. The strains used in this study are listed in Table 1.

TABLE 1

| | Parent | | |
|---|---|---|---|
| Strain | strain | Genotype/plasmid ID | Selection |
| LL1299 | DSM | DSM 1313 Δhpt Δ0478 (laboratory | — |
| | 1313 | evolved) | |
| AG8235 | LL1299 | DSM 1313 Δhpt Δ0478 2366::poly- | — |
| | | attB/pNA24D | |
| AG13738 | AG8235 | DSM 1313 Δhpt Δ0478 2366::pYK- | Tm15 |
| | | NA18/pYK-NA18 | μg/ml |

Plasmid Construction and sequencing confirmations

The plasmids used in this study are listed in Table 2 and the plasmid DNA sequences are given in the “DNA sequences” section. The plasmids were computationally designed, and the DNA synthesis and cloning services of Genscript, Inc. were used for their construction.

TABLE 2

| Plasmid | | | | Selectable |
|---|---|---|---|---|
| ID | Cargo | Use | Origin^{organism} | Marker^{conc(ug/mL)} |
| pNA24D | Clo1313_2366 | Replaces Clo1313_2366 gene with | p15A^{E. coli} | Tm¹⁵ |
| | Homologies | poly-attB cassette (landing pad) | pNW33N^{C. thermocellum} | |
| | flanking | using homologous recombination | | |
| | poly-attB | | | |
| | cassette | | | |
| pNA42 | P2366: | Expresses the Y412MC61 serine | p15A^{E. coli} | Neo³⁰⁰ |
| | Y412MC61 | recombinase. Co- transformed with | pNW33N^{C. thermocellum} | |
| | | cargo | | |
| | | containing Y412MC61 attP site | | |
| pYK- | Pate_0603: | Cargo plasmid that expresses sfGFP | ColE1^{E. coli} | Tm¹⁵ |
| NA18 | sfGFP; | driven by the xylose-inducible | | |
| | Pctx_1194: | promoter and xylR | | |
| | Athe_0617 | | | |
| | (xlyR) | | | |

Competent cell preparation and transformation

Plasmids were transformed and propagated in our in-house E. coli Top10 Adcm methylase-deficient strain (Top10DCM-) before transformation into C. thermocellum competent cells. Top10DCM-cells were grown in LB overnight in a shaking incubator at 37° C. The overnight grown culture was used for inoculating 50 ml of LB in a 250 ml flask to an OD600 of ˜ 0.05. The culture was then grown for ˜2-3 hours in a shaking incubator at 37° C. to a final OD600 between 0.5 and 0.7. The cells were spun down at 6000 RPM in a chilled centrifuge, washed thrice with ice-cold 10% glycerol solution, and the pellet was resuspended in 500 μL of 10% glycerol. 30 μL aliquots of the Top10DCM-electro-competent cells were either stored at −80° C. for later use or immediately used to transform plasmids. The E. coli pre-set protocol (Exponential decay, 25 μF, 200 ohm, 1800 volts, 0.1 cm cuvette) in the Bio-Rad Gene Pulser Xcell was used for transforming the plasmids into Top10DCM-electro-competent cells. After the electrical pulsing, the cells in the cuvette were resuspended in 950 μL of SOC medium, incubated at 30° C. for 3-4 hours, and plated on LB with 50 μg/ml of chloramphenicol and incubated at 30° C. overnight. The colonies were confirmed by PCR for successful plasmid transformation. Plasmids were extracted using a Zymo Research midi prep kit from 50 ml liquid cultures (LB with appropriate antibiotics) using the low copy number plasmid extraction protocol.

C. thermocellum competent cells were made using an already-established protocol briefly explained here. A frozen stock of C. thermocellum stored at −80° C. was used to inoculate 5 ml of CTFUD (with or without antibiotics), which was grown at 50° C. inside the Coy anaerobic chamber without shaking. The 5 ml seed culture was used to inoculate 500 ml of CTFUD (with or without appropriate antibiotics) and grown overnight at 50° C. in the anaerobic Coy chamber to an OD600 between 0.5 and 0.7. The culture was then chilled on ice, spun down aerobically at 6000 RPM for 15 minutes on a benchtop centrifuge set at 19° C., and washed with ice-cold electroporation buffer (250 mM sucrose and 10% glycerol) three times. The pellet from a 500 ml culture was resuspended in 100 μL of fresh, sterile electroporation buffer. The cells were either used immediately for transformation or were stored at −80° C. for use later. A square wave protocol (1000 V, 1.5 msec, 1 pulse, 1 mm cuvette) in the Bio-Rad Gene Pulser Xcell was used for transforming the plasmids into C. thermocellum using 30 μL of competent cells and 1 μg of each plasmid. After the electrical pulsing, the cells were resuspended in 1 ml of CTFUD (no antibiotic) and recovered at 50° C. overnight inside the anaerobic Coy chamber. The recovered culture was plated on CTFUD-agar with appropriate antibiotics. The colonies formed were picked into CTFUD with appropriate antibiotics and then confirmed by PCR. The primers used in this work for PCR confirmation of integration are listed in Table 3.

TABLE 3

| Primer | SEQ ID NO: | Sequence |
|---|---|---|
| #10 | 29 | cggtttctttaccagact |
| #11 | 30 | ggtattttacccttgtcgtt |
| #13 | 31 | ctctgctactcgtttttcaatc |
| #14 | 32 | gggaaggatttgaggttagg |
| #49 | 33 | ggcaccaataactgccttaaaga |
| #50 | 34 | cgatgataaacaaaggacggttc |

Chromosomal insertion of the xylose-inducible promoter

The xylose-inducible expression cassette was inserted into the C. thermocellum chromosome by co-transforming 1 μg of the integrating plasmid (pYK-NA18, Table 2) and 1 μg of the helper plasmid (pNA42, Table 2) by electroporation into competent cells of C. thermocellum AG8235. After an overnight anaerobic recovery at 50° C., the cells were plated on CTFUD-agar with 15 μg/ml of thiamphenicol. Colonies typically appeared in 2-3 days after incubation at 50° C. Colonies were picked into liquid CTFUD with 15 μg/ml of thiamphenicol, and subsequently, confirmed by PCR using primers #13 and #14, listed in Table 3.

Promoter characterization using sfGFP assay

Promoter activity was measured by following fluorescence of sfGFP. For the sfGFP assay, the strains were cultured in liquid CTFUD supplemented with both 15 μg/ml of thiamphenicol and D-(+)-xylose (3.125, 6.25, 12.5, 25, and 50 mM; Sigma-Aldrich, X1500-500G) at 50° C. for 48 hours. The induced cultures were washed three times with 1×PBS and resuspended in 1 ml of 1×PBS for overnight incubation to allow sfGFP folding in the dark. The OD600-normalized fluorescence of the cultures was measured by excitation at 488 nm and emission at 510 nm in a BioTek fluorescence plate reader.

Sequences

SEQ ID NO: 1
XylR of expression cassette
Artificial
Nucleic Acid
TCATGCATATTCTGGAAAAGAAAGCATGTCGGAAATCACCATAGCTATACATCCCAG
CACCACAGCTCTGTCTTTCAGTTTTGAAACCTCTATCTTAAGATTATAAAACTGGGCG
ATAAAGGATCTCTGGTTAATTACTTCCCTCAATTTTTCTAAAAACAACTCTCCAAAA
AATGATGCCTTATTACCTATAATTACCATTTCAGGATTAAAAATATTTACAAGATTTG
CCACGCCTATCCCCATCTTTTCAGCAACCTCAAGTATAGCCATTCTGCAAACTCTGCT
TCCTTCTTTTGCTGCTTGTATTATCCGAGAAGGAGTGATTTCATCTACATTTTCCCAG
CTTATATACCTATCCTCTACTCCTTGTTTTACAAGCTTTTTTATAACACTCAAAAGTG
CCCTCTCGGATGCAAAGTTCTCAAGACAGCCAATATTGCCGCAGCTGCAAACATCGT
CCTGAAAGTTGATAGTGGTATGTCCAACTTCACCTGCAAATCCTGCAGCACCTCTGA
AAAGTTTGTTGTCGATAATAATTCCTGCACCAAGCCCAATTCCAACACTCAAATAAA
TCAAATCACTAACTTTTCCCCACTCACCAAACCACTTTTCGCCCAGTGCGCCTGCATT
TGCTTCATTGTCAATATAAACAGGGAGGTTGAACTTTTGCTGAACAATTGACCTCAA
AGGGACATTTTGCCATTTCAAATTGGGAGCAAGAAGGACAGTTCCAGACTCTTTTTC
TATAATACCTGGAACACCAATTCCAATACCTAAAATCCCTTTTGGAGTTTGTGGCGC
CTTTTTTACTGATTTTTCAATCAGGTCAAAAAGAAGCCTTAAAAGTTTTTCCTTATCC
TCACCTATTTTCATGTTGGCATATTCTTCAAAAATAACTTCTCCCACAAAGTTTGAGA
GAATAATATGAATATAGTCAACACCTAAGTCAATTCCGATGATTGAACCTACATCTT
TGTTTACCTGTAAAAGGACAGGTCTTCTTCCGCCTTTGGACTTACCGTATCCTTTTTC
TACTACATACCCTTCTTTTATGAGCTCATCGGTGAGGTTTGAGACTGTTGCTTTATTT
AAATCTACAAGTTTCGATATCTTTGTACGAGATATTATCTTGTTGTCCAAAATTGTTT
TCAAAACCAAAAGCTTGTTTATTTGCTTTAGTAGCGTGTGGTTACCCATAG
SEQ ID NO: 2
XylR binding site
Artificial
Nucleic Acid
GTTTGTTTAATAAACAAAACTAAG
SEQ ID NO: 3
Pctx_1194 of expression cassette
Artificial
Nucleic Acid
TTTTTTTCCCCCTTTAATGATAATTAAGATATTGCTTATACAATAAATTATATTATAA
ATTAATCTAAAAAAAAACAACATGAAATAATTACTATTTTTAGTGAAAATATTACGT
TACAACTCAAATTGCGCCGTTTTGCTTATCCTACGTCAGTTTTTTAGCTTATTGTATT
AATTTTACCGGGAGGCAAAATATACCGCTGCCTGAAACATTTATAGCCAATACAGTT
ATCTTGCCTGTTTCGTCGGCTGCAAAATGCATTATTTGACGGGAATTGCAACGTGGT
CGGCATATTAATGCAAGTTGAACTGACAATCTGCCATTATTTTTTGCAAAATAATCTT
TGTCTTGA
SEQ ID NO: 4
Pate_0603 of expression cassette
Artificial
Nucleic Acid
AAGAAGGATTTTTTGAAAAAATGAAGAATATATATTATAGATATTAGTTTGTTTAAT
AAACAAACTAAGTACACGTACTGGCATGTTTAAAAAATAAGGTTTAGTTAAAAAAC
TGATTTATTATAGAAGGAGAGTGAGTTATAAAATGCGTAAAGGCGAAGAGCTGTTC
SEQ ID NO: 5
sfGFP of expression cassette
Artificial
Nucleic Acid
ACTGGTGTCGTCCCTATTCTGGTGGAACTGGATGGTGATGTCAACGGTCATAAGTTT
TCCGTGCGTGGCGAGGGTGAAGGTGACGCAACTAATGGTAAACTGACGCTGAAGTT
CATCTGTACTACTGGTAAACTGCCGGTACCTTGGCCGACTCTGGTAACGACGCTGAC
TTATGGTGTTCAGTGCTTTGCTCGTTATCCGGACCATATGAAGCAGCATGACTTCTTC
AAGTCCGCCATGCCGGAAGGCTATGTGCAGGAACGCACGATTTCCTTTAAGGATGA
CGGCACGTACAAAACGCGTGCGGAAGTGAAATTTGAAGGCGATACCCTGGTAAACC
GCATTGAGCTGAAAGGCATTGACTTTAAAGAAGACGGCAATATCCTGGGCCATAAG
CTGGAATACAATTTTAACAGCCACAATGTTTACATCACCGCCGATAAACAAAAAAAT
GGCATTAAAGCGAATTTTAAAATTCGCCACAACGTGGAGGATGGCAGCGTGCAGCT
GGCTGATCACTACCAGCAAAACACTCCAATCGGTGATGGTCCTGTTCTGCTGCCAGA
CAATCACTATCTGAGCACGCAAAGCGTTCTGTCTAAAGATCCGAACGAGAAACGCG
ATCATATGGTTCTGCTGGAGTTCGTAACCGCAGCGGGCATCACGCATGGTATGGATG
AACTGTACAAA
SEQ ID NO: 6
Terminator of expression cassette
Artificial
Nucleic Acid
GACGAACAATAAGGCCTCCCTAACGGGGGGCCTTTTT TATTGATAACAAAA
SEQ ID NO: 7
Xylose inducible sfGFP expression cassette
TCATGCATATTCTGGAAAAGAAAGCATGTCGGAAATCACCATAGCTATACATCCCAG
CACCACAGCTCTGTCTTTCAGTTTTGAAACCTCTATCTTAAGATTATAAAACTGGGCG
ATAAAGGATCTCTGGTTAATTACTTCCCTCAATTTTTCTAAAAACAACTCTCCAAAA
AATGATGCCTTATTACCTATAATTACCATTTCAGGATTAAAAATATTTACAAGATTTG
CCACGCCTATCCCCATCTTTTCAGCAACCTCAAGTATAGCCATTCTGCAAACTCTGCT
TCCTTCTTTTGCTGCTTGTATTATCCGAGAAGGAGTGATTTCATCTACATTTTCCCAG
CTTATATACCTATCCTCTACTCCTTGTTTTACAAGCTTTTTTATAACACTCAAAAGTG
CCCTCTCGGATGCAAAGTTCTCAAGACAGCCAATATTGCCGCAGCTGCAAACATCGT
CCTGAAAGTTGATAGTGGTATGTCCAACTTCACCTGCAAATCCTGCAGCACCTCTGA
AAAGTTTGTTGTCGATAATAATTCCTGCACCAAGCCCAATTCCAACACTCAAATAAA
TCAAATCACTAACTTTTCCCCACTCACCAAACCACTTTTCGCCCAGTGCGCCTGCATT
TGCTTCATTGTCAATATAAACAGGGAGGTTGAACTTTTGCTGAACAATTGACCTCAA
AGGGACATTTTGCCATTTCAAATTGGGAGCAAGAAGGACAGTTCCAGACTCTTTTTC
TATAATACCTGGAACACCAATTCCAATACCTAAAATCCCTTTTGGAGTTTGTGGCGC
CTTTTTTACTGATTTTTCAATCAGGTCAAAAAGAAGCCTTAAAAGTTTTTCCTTATCC
TCACCTATTTTCATGTTGGCATATTCTTCAAAAATAACTTCTCCCACAAAGTTTGAGA
GAATAATATGAATATAGTCAACACCTAAGTCAATTCCGATGATTGAACCTACATCTT
TGTTTACCTGTAAAAGGACAGGTCTTCTTCCGCCTTTGGACTTACCGTATCCTTTTTC
TACTACATACCCTTCTTTTATGAGCTCATCGGTGAGGTTTGAGACTGTTGCTTTATTT
AAATCTACAAGTTTCGATATCTTTGTACGAGATATTATCTTGTTGTCCAAAATTGTTT
TCAAAACCAAAAGCTTGTTTATTTGCTTTAGTAGCGTGTGGTTACCCATAGTTTTTTT
CCCCCTTTAATGATAATTAAGATATTGCTTATACAATAAATTATATTATAAATTAATC
TAAAAAAAAACAACATGAAATAATTACTATTTTTAGTGAAAATATTACGTTACAACT
CAAATTGCGCCGTTTTGCTTATCCTACGTCAGTTTTTTAGCTTATTGTATTAATTTTAC
CGGGAGGCAAAATATACCGCTGCCTGAAACATTTATAGCCAATACAGTTATCTTGCC
TGTTTCGTCGGCTGCAAAATGCATTATTTGACGGGAATTGCAACGTGGTCGGCATAT
TAATGCAAGTTGAACTGACAATCTGCCATTATTTTTTGCAAAATAATCTTTGTCTTGA
AAGAAGGATTTTTTGAAAAAATGAAGAATATATATTATAGATATTAGTTTGTTTAAT
AAACAAACTAAGTACACGTACTGGCATGTTTAAAAAATAAGGTTTAGTTAAAAAAC
TGATTTATTATAGAAGGAGAGTGAGTTATAAAATGCGTAAAGGCGAAGAGCTGTTC
ACTGGTGTCGTCCCTATTCTGGTGGAACTGGATGGTGATGTCAACGGTCATAAGTTT
TCCGTGCGTGGCGAGGGTGAAGGTGACGCAACTAATGGTAAACTGACGCTGAAGTT
CATCTGTACTACTGGTAAACTGCCGGTACCTTGGCCGACTCTGGTAACGACGCTGAC
TTATGGTGTTCAGTGCTTTGCTCGTTATCCGGACCATATGAAGCAGCATGACTTCTTC
AAGTCCGCCATGCCGGAAGGCTATGTGCAGGAACGCACGATTTCCTTTAAGGATGA
CGGCACGTACAAAACGCGTGCGGAAGTGAAATTTGAAGGCGATACCCTGGTAAACC
GCATTGAGCTGAAAGGCATTGACTTTAAAGAAGACGGCAATATCCTGGGCCATAAG
CTGGAATACAATTTTAACAGCCACAATGTTTACATCACCGCCGATAAACAAAAAAAT
GGCATTAAAGCGAATTTTAAAATTCGCCACAACGTGGAGGATGGCAGCGTGCAGCT
GGCTGATCACTACCAGCAAAACACTCCAATCGGTGATGGTCCTGTTCTGCTGCCAGA
CAATCACTATCTGAGCACGCAAAGCGTTCTGTCTAAAGATCCGAACGAGAAACGCG
ATCATATGGTTCTGCTGGAGTTCGTAACCGCAGCGGGCATCACGCATGGTATGGATG
AACTGTACAAATGATCTAGAGACGAACAATAAGGCCTCCCTAACGGGGGGCCTTTTT
TATTGATAACAAAA
SEQ ID NO: 8
Athe_0603
Caldicellulosiruptor bescii
Nucleic Acid
TGCA AGTTGAACTG ACAATCTGCC ATTATTTTTT GCAAAATAAT CTTTGTCTTG
AAAGAAGGAT TTTTTGAAAA AATGAAGAAT ATATATTA TAGATATTAG
TTTGTTTAAT AAACAAACTA AGTACACGTA CTGGCATGTT TAAAAAATAA
GGTTTAGTTA AAAAACTGAT TTATTATAG AAGGAGAGTG AGTTATAAA
SEQ ID NO: 9
Athe_0603
Caldicellulosiruptor bescii
Amino Acid
MKYFKDIPEVKYEGPQSDNPFAFKYYNPDEIIDGKPLKDHLRFAIAYWHTFCATGSDPFG
QPTIVRPWDKFSNRMDNAKARVEAAFEFFELLDVPFFCFHDRDIAPEGENLKESNKNLD
EIVSLIKEYLKTSKTKVLWGTANLFSHPRYVHGAATSCNADVFAYAAAQVKKALEVTK
ELGGENYVFWGGREGYETLLNTDMGLELDNLARFLHMAVEYAKEIGFDGQFLIEPKPK
EPTKHQYDFDSAHVYGFLKKYDLDKYFKLNIEVNHATLAGHDFHHELRFARINNMLGSI
DANMGDLLLGWDTDQFPTDVRLTTLAMYEVIKAGGFDKGGLNFDAKVRRGSFELEDL
VIGHIAGMDAFAKGFKIAYKLVKDGVFDKFIDERYKSYKEGIGAKIVSGEANFKMLEEY
ALSLDKIENKSGKQELLEMILNKYMFSE
SEQ ID NO: 10
Calkr_1977
Caldicellulosiruptor acetigenus I77R1B
Nucleic Acid
TCA AGTTGAACTA ATGGCGTGCT ATTTTCTTTG GAAAAAATTT TTTTGCTTTA
AAAGAAGGAT TTTTTGAAAA AATGAAGAAT ATATATTATA ATGATGTTAG
TTTGTTTAAT AAACAAACTA ATTGTATACA AGAATTTTGT TTAAAACTTA
GATGTGCTTA AAAAATTTTT CATTATAG AAGGAGAGTG AGTTATAAA
SEQ ID NO: 11
SOJ16_000578
Caldicellulosiruptor danielii
Nucleic Acid
CA AGTTGAACTG ACAATCTGCC ATTA TTTTT GCAAAATAAT CTTTGTTGTG
AAAGAAGGAT TTTTTGAAAA AATGAAGAAT ATATATTATG ATGATGTTAG
TTTGTTTAAT AAACAAACTA AGTATACGTA CTGCATGTT CTAAATTAA
GGTTTAGTTT AAAAATTACT TTTATTATAG AAGGAGAGTG AGTTATAAA
SEQ ID NO: 12
Calhy_2055
Caldicellulosiruptor hydrothermalis
Nucleic Acid
A AGTTGAACTG ACAATCTGCC ATTA TTTTT GCAAAATAAT CTTTGTTGTG
AAAGAAGGAT TTTTTGAAAA AATGAAGAAT ATATATTATA ATGATGTTAG
TTTGTTTAAT AAACAAACTA AGTACGCGTA CTGGCATGTT TAAAAAATAA
GGTTTAGTTA AAAAAATTAT TT ATTATAG AAGGAGAGTG AGTTATAAA
SEQ ID NO: 13
Calkro_2027
Caldicellulosiruptor kronotskyensis
Nucleic Acid
GCACA AGTTTAATTG ACAATATGCC TTT ATTTT TAAAAACATT CTTTGTTTTG
AAAGAAGGAT TTTTTGAAAA AGTGGAGAAT ATATATTATA ATAAACTTAG
TTTGTTTAAT AAACAAACTA AGTATGTGTA TGGCATGTT TAA AAATAA
GATTTAGTCA AAAAATTATA T ATTATAG AAGGAGAGTG AGTTATAAA
SEQ ID NO: 14
COB47_0553
Caldicellulosiruptor obsidiansis
Nucleic Acid
TCATACA AGTTAAATTA A_ TAGT CTGCTACTTT TGCAAAACAT TCTTTGTTTT
AAAGAAGGAT TTTTTGAAAA AGTGGAGAAT ATATATTATA ATAAACTTAG
TTTGTTTAAT AAACAAACTA AGTATGTTTA CAAACATTTT TAAAAAAGTG
GGTTTACGTT AAGAAATTA TTTATTATAG AAGGAGAGTG AGTTATAAA
SEQ ID NO: 15
Calow_0468
Caldicellulosiruptor owensensis
Nucleic Acid
TTAAAATGCA AGTTGAACTG ACAATCTGCT AT TTTTTGG CAAAAAATTT
TTTTGCTTTA AAAGAAGGAT TTTTTGAAAG GATGAAGAAT ATA TT
ATAATGTTAG TTTGTTTAAT AAACAAACTA ATTGTATGTA AGGACTTTGT
TTAAAACTTA GATGTGCTTA AAAATTTTTT ATTATAG AAGGAGAGTG
AGTTATAAA
SEQ ID NO: 16
Athe_0617
Caldicellulosiruptor bescii
Amino Acid
MGNHTLLKQINKLLVLKTILDNKIISRTKISKLVDLNKATVSNLTDELIKEGYVVEKGYG
KSKGGRRPVLLQVNKDVGSIIGIDLGVDYIHIILSNFVGEVIFEEYANMKIGEDKEKLLR
LLFDLIEKSVKKAPQTPKGILGIGIGVPGIIEKESGTVLLAPNLKWQNVPLRSIVQQKFNL
PVYIDNEANAGALGEKWFGEWGKVSDLIYLSVGIGLGAGIIIDNKLFRGAAGFAGEVGH
TTINFQDDVCSCGNIGCLENFASERALLSVIKKLVKQGVEDRYISWENVDEITPSRIIQA
AKEGSRVCRMAILEVAEKMGIGVANLVNIFNPEMVIIGNKASFFGELFLEKLREVINQRS
FIAQFYNLKIEVSKLKDRAVVLGCIAMVISDMLSFPEYA
SEQ ID NO: 17
Csac_0695
Caldicellulosiruptor saccharolyticus
Amino Acid
MGNHTLLKQINKLLVLKTILDNKTISRAKISKLVDLNKATVSNLTDELIKEGFVIEKGYG
HSKGGRRPVLLEVNKNVGLIIGIDLGVNYIHLILTNFIGEIVWEKSANIRLGETQERILEVL
FELIGEAIKVAPQTQKGILGIGIGVPGIVEKTSGIVLIAPNLRWKDVPLKAMVEERFNLP
VYIDNEANAGALGEKWFGNWGDVSHLVYVSVGIGIGAGIVIGDEIYRGAKGFAGEVGH
TTIDFNDDVCSCGNVGCLENFASERALLSLIQKIVESGWEDEYINKKNVDKLDASYIIDS
AKLGSKVALHAIKDIANKLGIGIANLVNIFNPDIVIIGNKASFMGDLFLEELRRIVFKRS
FITQYHHVRVEISKLKDRACVLGCVAMVISDMLAFPDYV
SEQ ID NO: 18
COB47_0572
Caldicellulosiruptor obsidiansis,
Amino Acid
MGNHALLKQINKLLILKTILDNKMISRAKISRLVDLNKATVSNLTDELIKEGYIVEKGYG
KSKGGRRPVLLQVNKDVGSIIGIDLGVDYIHVILSNFIGEIIFEEYVNIKMEEPKEKLLN
LLFDMIEKAIDKAPPTPKGILGIGIGVPGIVEKESGIVLIAPNLKWKNVHLKSIIEQRFNL
PVYIDNEANAGALGEKWFGEWGKVSDLIYLSVGIGLGAGIIIDNKLFRGAAGFAGEVGH
TTINFQDDVCSCGNIGCLENFASERALLSVIKKLVKEGAEDRYISCENVDEITPSQIIQA
AMDGSRICRMAVLEVAEKMAIGIANLVNIFNPEIVIVGNKASFFGDLFLEKLREVVNQKS
FIAQFYDLKIEVSKLKDRAVVLGCIAMVISDMLSFPEYV
SEQ ID NO: 19
Calhy_2039
Caldicellulosiruptor hydrothermalis
Amino Acid
MGNHTLLKQINKLLVLKTILDNKIISRAKISRLVDLNKATVSNLTDELIKEGYVVEKGYG
KSKGGRRPVLLQVNKDVGSIIGIDLGVDYIHIILSNFVGEVIFEEYANMKMGEDKEKLLD
LLFDLIERAINRAPQTPKGILGIGIGVPGIVEKESGIVLIAPNLKWKNVHLKSIVQQRFNL
PVYIDNEANAGALGEKWFGEWGKVTDLIYLSVGIGLGAGIIIDNKLFRGAAGFAGEVGH
TTINFQDDVCSCGNIGCLENFASERALLSVIKKLVKEGAEDRYISCENVDEITPSQIIQA
AKDGSRVCRMAVLEVAEKMAIGIANLVNIFNPEIVIIGNKVSFFGDLFLEKLREVVNQKS
FIAQFYDLKIEVSKLKDRAVVLGCIAMVISDMLSFPEYT
SEQ ID NO: 20
Calow_0493
Caldicellulosiruptor owensensis
Amino Acid
MGNHALLKQINKLLILKTILDNKMISRAKISRLVDLNKATVSNLTDELIKEGYIVEKGYG
KSKGGRRPVLLQVNKDVGSIIGIDLGVDYIHVILSNFIGEIIFEEYVNIKMKEPKEKLLN
LLFDMIEKAIDKAPPTPKGILGIGIGVPGIVEKESGIVLIAPNLEWKNVHLKSIIEQKFNL
PVYIDNEANAGALGEKWFGEWGKVSDLIYLSVGIGLGAGIIIDNKLFRGAAGFAGEVGH
TTINFQDDVCSCGNIGCLENFASERALLSIIKKLVKEGAEDRYISCENVDEITPSQIIQA
AMDGSRICRMAVLEVAEKMAIGIANLVNIFNPEIVIVGNKASFFGDLFLEKLREAVNQKS
FIAQFYDLKIEVSKLKDRAVVLGCIAMVISDMLSFPEYA
SEQ ID NO: 21
Calkr_0571
Caldicellulosiruptor acetigenus I77R1B
Amino Acid
MGNHSLIKQINKLLILKTILDKGVISRAKISRLVDLNKATVSNLTDELIKEGYVIEKGYG
KSKGGRRPVLLQVNKDVGSIIGIDLGVDYIHIILSNFIGEIIFEEYVNLKIKEDNEKFLN
IFFDLIQKAIDKAPETPKGILGIGIGVPGIVEKESGVVLVAPNLKWSNVPLKDIVQQRFNL
PVYIDNEANAGALGEKWFGEWGKVTDLIYLSVGIGLGAGIIIDNKLFRGAAGFAGEVGH
TTINFQDDVCSCGNIGCLENFASERALLSVIKKLVKEGAEDRYISCENIDEITPSQIIQAA
KDGSRVCRMAVLEVAEKMGIGVANLVNIFNPEIVIIGNKASFFGDLFLEKLREVVNQRS
FIAQFYNLKIEVSKLKDRAVVLGCIAMVISDMLSFPEYT
SEQ ID NO: 22
Calkro_2009
Caldicellulosiruptor kronotskyensis
Amino Acid
MGNHTLLKQINKLLVLKTILDNKIISRAKISRLVDLNKATVSNLTDELIKEGYIVEKGYG
KSKGGRRPVLLQVNKDVGSIIGIDLGVDYIHVILSNFVGEVIFEEYAEMKMGEDKDKLFD
LLFDLIEKAIDRAPHTPKGILGIGIGVPGIVEKESGIVLIAPNLKWKNVHLKSIVQQRFNL
PVYIDNEANAGALGEKWFGEWGKVSDLIYLSVGIGLGAGIIIDNKLFRGAAGFAGEVGH
TTINFQDDVCSCGNIGCLENFASERALLSVIKKLVKQGVEDRYISCENVHEITPSRIIQA
AKEGSRVCRMAILEVAEKMGIGVANLVNIFNPEMVIIGNKASFFGELFLEKLREVINQRS
FIAQFYNLKIEVSKLKDRAVVLGCIAMVISDMLSFPEYA
SEQ ID NO: 23
Calla_1782
Caldicellulosiruptor acetigenus 6A
Amino Acid
MGNHSLIKQINKLLILKTILDKGVISRAKISRLVDLNKATVSNLTDELIKEGYVIEKGYG
KSKGGRRPVLLQVNNDVGSIIGIDLGVDYIHIILSNFIGEIIFEEYVNLKIKEDNEKFLNIF
FDLIQKAIDKAPETPKGILGIGIGVPGIVEKESGVVLVAPNLKWSNVPLKDIVKQRFNL
PVYIDNEANAGALGEKWFGEWGKVTDLIYLSVGIGLGAGIIIDNKLFRGAAGFAGEVGH
TTINFQDDVCSCGNIGCLENFASERALLSVIKKLVKEGAEDRYISCENIDEITPSQIIQA
AKDGSRVCRMAVLEVAEKMGIGVANLVNIFNPEIVIIGNKASFFGDLFIEKLREVVNQRS
FIAQFYNLKIEVSKLKDRAVVLGCIAMVISDMLSFPEYA
SEQ ID NO: 24
ELD05_11440
Caldicellulosiruptor changbaiensis
Amino Acid
MGNHTLLKQINKLLVLKTILDNKTISRAKISRLVDLNKATVSNLTDELIREGFVIEKGYG
HSKGGRRPVLLEVNKNVGLIIGIDLGVNYIHLVLTNFIGEIVWEKSANIRLGETQERILEV
LFELISEAIKVAPQTQKGILGIGIGVPGIVEKTSGTVLIAPNLRWKDVPLKAMVEERFNLP
VYIDNEANAGALGEKWFGDWGDVSHLVYVSVGIGIGAGIVIGDEIYRGAKGFAGEVGH
TTIDFNDDVCSCGNVGCLENFASERALLSLIQKMVESSWEDEYINKKNVDKLDASYIIDS
AKLGSKVALHAIKDIANKLGIGIANLVNIFNPDIVIIGNKASFMGDLFLEELRRIVFKRS
FITQYHHVRVEVSRLKDRACVLGCVAMVISDMLSFPDYV
SEQ ID NO: 25
OTJ99_000465
Caldicellulosiruptor naganoensis
Amino Acid
MGNHTLLKQINKLLVLKTILDNKTISRAKISRLVDLNKATVSNLTDELIREGFVIEKGYG
HSKGGRRPVLLEVNKNVGLIIGIDLGVNYIHLVLTNFIGEIVWEKSANIRLGETQERILEIL
FELISDAIKVAPQTQKGILGIGIGVPGIVEKTSGTVLIAPNLRWKDVPLKTMVEEKENLP
VYIDNEANAGALGEKWFGDWGDVSHLVYVSVGIGIGAGIVIGDEIYRGAKGFAGEVGH
TTIDFNDDVCSCGNVGCLENFASERALLSLIQKIVESGWEDEYINKKNVDKLDASYIIDS
AKLGSKVALHAIKDIANKLGIGIANLVNIFNPDIVIIGNKASFMGDLFLEELRRIVFKRS
FITQYHRVRVEVSRLKDRACVLGCVAMVISDMLAFPDYV
SEQ ID NO: 26
OTK00_002089
Caldicellulosiruptor morganii
Amino Acid
MGNHALIKQINKLLILKTILDHRTISRAKISRLVDLNKATVSNLTDELIKEGFVIEKGYGH
SKGGRRPVLLEVNKNVGLIIGIDLGVNYIHLILTNFIGEIIWERNMAIKLGEKQEKILESL
FMIIEEAIKAAPPTQKGILGIGIGVPGIVEKNSGTVLLAPNLKWQDVPLKRMVESRFGLP
VYIDNEANAGALGEKWFGGWGDVSHLLYVSVGIGIGAGIVIGEEVYRGAKGFAGEVGH
MTIDENDDVCSCGNVGCLENFASERALLSLIKTLVESGVEDEYINRDTVEEMDAGYIIQS
ALQRSRVAMNAITDIANKLGIGIANLVNIFNPDIVVIGNKASFMGDLFLEELRRIVYKRA
FITQYHHVRVEVSKLKDRACVLGCVAMVISDMLAFPDYV
SEQ ID NO: 27
SOJ16_000597
Caldicellulosiruptor danielii
Amino Acid
MGNHSLIKQINKLLILKTILDHKVISRAKISRLVDLNKATVSNLTDELIKEGYVIEKGYG
KSKGGRRPVLLQVNKDVGSIIGIDLGVDYIHLILSNFVGEIIFEEYVNLRFGENKEKFLN
TLFALIQKAIDKSPQTPKGILGIGIGVPGIVEKESGVVLIAPNLKWVNIPLKEILEDRFKL
PVYIDNEANAGALGEKWFGEWGKVSDLIYLSVGIGLGAGIIIDNKLFRGAAGFAGEVGH
TTINFQDDVCSCGNIGCLENFASERALLSVIKKLVKEGTEDRYVSCENVDEITPSQIIQA
AKDGSRVCRMAVLEIAEKMGIGVANLVNIFNPEIVIIGNKASFFGDLFLEKLREVVNQKS
FIAQFYNLKIEVSKLKDKAVVLGCIAMVISDMLSFPEYA
SEQ ID NO: 28
CaldiYA01_18370
Caldicellulosiruptor diazotrophicus
Amino Acid
MGNHTLLKQINKLLVLKTILDNKIISRAKISRLVDLNKATVSNLTDELIKEGYIVEKGYG
KSKGGRRPVLLQVNKDVGSIIGIDLGVDYIHIILSNFVGEIIFEEYADIKMGENKEKLFE
LLFELIEKAIDKSPQTPKGILGIGIGVPGIVEKKSGIVLIAPNLKWKNVHLKSIVQQRFNL
PVYIDNEANAGALGEKWFGEWGKVSDLIYLSVGIGLGAGIIIDNKLFRGAAGFAGEVGH
TTINFQDDVCSCGNIGCLENFASERALLSVIKKLVKEGAEDRYISCENVDEITPFQIIQA
AMDGSRVCRMAVLEVAEKMAIGIANLVNIFNPEIVIIGNKASFFGDLFLEKLREIVNQKS
FIAQFYNLKIEVSKLKDRAVVLGCIAMVISDMLSFPEYA

1. A combination of a first expression cassette and a second expression cassette, wherein the first expression cassette comprises a heterologous promoter operably linked to a nucleic acid encoding a regulatory protein, wherein the regulatory protein is a xylose regulator and a homolog of xyIR; and
wherein the second expression cassette comprises an inducible promoter operably linked to a heterologous nucleic acid sequence for a gene of interest, wherein activity of the inducible promoter is modulated in a thermophile by the regulatory protein in the first expression cassette.

2. The combination of claim 1, wherein the inducible promoter comprises a nucleic acid sequence as set forth in SEQ ID NO: 2.

3. The combination of claim 2, wherein the inducible promoter comprises a nucleic acid sequence comprising at least 80% sequence identity to SEQ ID NO: 1.

4. The combination of claim 3, wherein the inducible promoter comprises a nucleic acid sequence as set forth in SEQ ID NO: 1.

5. The combination of claim 1, wherein the regulatory protein is a xylose regulator from a Caldicellulosiruptor bacterium.

6. The combination of claim 1, wherein the regulatory protein is encoded by a xylR from a Caldicellulosiruptor bacterium.

7. The combination of claim 5, wherein the Caldicellulosiruptor bacterium is a C. bescii, C. saccharolyticus, C. obsidiansis, C. hydrothermalis, C. owensensis, C. acetigenus, C. kronotskyensis, C. acetigenus, C. changbaiensis, C. naganoensis, C. morganii, C. danielii, or C. diazotrophicus bacterium.

8. The combination of claim 1, wherein the regulatory protein comprises an amino acid sequence comprising at least 90% sequence identity to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

9. The combination of claim 1, wherein the regulatory protein comprises an amino acid sequence as set forth in SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

10. The combination of claim 1, wherein the two expression cassettes are placed on the same vector.

11. The combination of claim 1, wherein the gene of interest is thermophilic Cas9 (GeoCas9), thermophilic Cas13 (TccCas13a), or encodes the protein of a thermostable beta-glucuronidase (gus-tr3337), the red fluorescent protein mScarlet3, the superfolder green fluorescent protein (sfGFP), pyruvate decarboxylase, or alcohol dehydrogenase.

12. The combination of claim 1, wherein the thermophile is Clostridium thermocellum, Parageobacillus thermoglucosidasius (previously called Geobacillus thermoglucosidasius), or Bacillus licheniformis.

13. A host cell comprising the combination of a first and a second expression cassettes of claim 1, wherein the host cell is a thermophilic bacterium.

14. The host cell of claim 13, wherein the inducible promoter comprises a nucleic acid sequence as set forth in SEQ ID NO: 2.

15. The host cell of claim 13, wherein the inducible promoter comprises a nucleic acid sequence with 80% sequence identity to SEQ ID NO: 1.

16. The host cell of claim 15, wherein the inducible promoter comprises a nucleic acid sequence as set forth in SEQ ID NO: 1.

17. The host cell of claim 13, wherein the regulatory protein is a xylose regulator from a Caldicellulosiruptor bacterium.

18. The host cell of claim 13, wherein the regulatory protein is encoded by a xylR from a Caldicellulosiruptor bacterium.

19. The host cell of claim 17, wherein the Caldicellulosiruptor is a C. bescii, C. saccharolyticus, C. obsidiansis, C. hydrothermalis, C. owensensis, C. acetigenus, C. kronotskyensis, C. acetigenus, C. changbaiensis, C. naganoensis, C. morganii, C. danielii, or C. diazotrophicus bacterium.

20. The host cell of claim 13, wherein the regulatory protein comprises an amino acid sequence comprising at least 90% sequence identity to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

21. The host cell of claim 13, wherein the regulatory protein comprises an amino acid sequence as set forth in SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

22. The host cell of claim 13, wherein the gene of interest is thermophilic Cas9 (GeoCas9), thermophilic Cas13 (TccCas13a) or encodes the protein of a thermostable beta-glucuronidase (gus-tr3337), the red fluorescent protein mScarlet3, the superfolder green fluorescent protein (sfGFP), pyruvate decarboxylase, or alcohol dehydrogenase.

23. The host cell of claim 13, wherein the second expression cassette is integrated into the genome of the host cell.

24. The host cell of claim 13, wherein the host cell is a Clostridium thermocellum, Parageobacillus thermoglucosidasius (previously called Geobacillus thermoglucosidasius), or Bacillus licheniformis.

25. A method of controlling gene expression in a thermophilic bacterium, the method comprising:
(a) obtaining a host cell according to claim 13, and
(b) incubating the host cell in the presence of xylose under conditions that result in the expression of the gene of interest.

26. The method of claim 25, wherein the inducible promoter comprises a nucleic acid sequence as set forth in SEQ ID NO: 2.

27. The method of claim 25, wherein the inducible promoter comprises a nucleic acid sequence comprising at least 80% sequence identity to SEQ ID NO: 1.

28. The method of claim 25, wherein the inducible promoter comprises a nucleic acid sequence as set forth in SEQ ID NO: 1.

29. The method of claim 25, wherein the regulatory protein is a xylose regulator from a Caldicellulosiruptor bacterium.

30. The method of claim 25, wherein the regulatory protein is encoded by a xylR from a Caldicellulosiruptor bacterium.

31. The method of claim 29, wherein the Caldicellulosiruptor bacterium is a C. bescii, C. saccharolyticus, C. obsidiansis, C. hydrothermalis, C. owensensis, C. acetigenus, C. kronotskyensis, C. acetigenus, C. changbaiensis, C. naganoensis, C. morganii, C. danielii, or C. diazotrophicus bacterium.

32. The method of claim 25, wherein the regulatory protein comprises an amino acid sequence comprising at least 90% sequence identity to SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

33. The method of claim 25, wherein the regulatory protein comprises an amino acid sequence as set forth in SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or SEQ ID NO: 28.

34. The method of claim 25, wherein the two expression cassettes are placed on the same vector.

35. The method of claim 25, wherein the gene of interest is thermophilic Cas9 (GeoCas9), thermophilic Cas13 (TccCas13a), or encodes the protein of a thermostable beta-glucuronidase (gus-tr3337), the red fluorescent protein mScarlet3, the superfolder green fluorescent protein (sfGFP), pyruvate decarboxylase, or alcohol dehydrogenase.

36. The method of claim 25, wherein the thermophile is Clostridium thermocellum, Parageobacillus thermoglucosidasius (previously called Geobacillus thermoglucosidasius), or Bacillus licheniformis.


SEQUENCE LISTING (34 sequences)

SEQ ID NO: 1 - DNA, 1202 bp, synthetic construct
  misc_feature 1..1202: note = Xylr of expression cassette
  source 1..1202: mol_type = other DNA
tcatgcatattctggaaaagaaagcatgtcggaaatcaccatagctatacatcccagcaccacagctctgtctttcagttttgaaacctctatcttaagattataaaactgggcgataaaggatctctggttaattacttccctcaatttttctaaaaacaactctccaaaaaatgatgccttattacctataattaccatttcaggattaaaaatatttacaagatttgccacgcctatccccatcttttcagcaacctcaagtatagccattctgcaaactctgcttccttcttttgctgcttgtattatccgagaaggagtgatttcatctacattttcccagcttatatacctatcctctactccttgttttacaagcttttttataacactcaaaagtgccctctcggatgcaaagttctcaagacagccaatattgccgcagctgcaaacatcgtcctgaaagttgatagtggtatgtccaacttcacctgcaaatcctgcagcacctctgaaaagtttgttgtcgataataattcctgcaccaagcccaattccaacactcaaataaatcaaatcactaacttttccccactcaccaaaccacttttcgcccagtgcgcctgcatttgcttcattgtcaatataaacagggaggttgaacttttgctgaacaattgacctcaaagggacattttgccatttcaaattgggagcaagaaggacagttccagactctttttctataatacctggaacaccaattccaatacctaaaatcccttttggagtttgtggcgccttttttactgatttttcaatcaggtcaaaaagaagccttaaaagtttttccttatcctcacctattttcatgttggcatattcttcaaaaataacttctcccacaaagtttgagagaataatatgaatatagtcaacacctaagtcaattccgatgattgaacctacatctttgtttacctgtaaaaggacaggtcttcttccgcctttggacttaccgtatcctttttctactacatacccttcttttatgagctcatcggtgaggtttgagactgttgctttatttaaatctacaagtttcgatatctttgtacgagatattatcttgttgtccaaaattgttttcaaaaccaaaagcttgtttatttgctttagtagcgtgtggttacccatag

SEQ ID NO: 2 - DNA, 24 bp, synthetic construct
  misc_feature 1..24: note = Xylr binding site
  source 1..24: mol_type = other DNA
gtttgtttaataaacaaaactaag

SEQ ID NO: 3 - DNA, 353 bp, synthetic construct
  misc_feature 1..353: note = Pctx_1194 of expression cassette
  source 1..353: mol_type = other DNA
tttttttccccctttaatgataattaagatattgcttatacaataaattatattataaattaatctaaaaaaaaacaacatgaaataattactatttttagtgaaaatattacgttacaactcaaattgcgccgttttgcttatcctacgtcagttttttagcttattgtattaattttaccgggaggcaaaatataccgctgcctgaaacatttatagccaatacagttatcttgcctgtttcgtcggctgcaaaatgcattatttgacgggaattgcaacgtggtcggcatattaatgcaagttgaactgacaatctgccattattttttgcaaaataatctttgtcttga

SEQ ID NO: 4 - DNA, 169 bp, synthetic construct
  misc_feature 1..169: note = Pate_0603 of expression cassette
  source 1..169: mol_type = other DNA
aagaaggattttttgaaaaaatgaagaatatatattatagatattagtttgtttaataaacaaactaagtacacgtactggcatgtttaaaaaataaggtttagttaaaaaactgatttattatagaaggagagtgagttataaaatgcgtaaaggcgaagagctgttc

SEQ ID NO: 5 - DNA, 690 bp, synthetic construct
  misc_feature 1..690: note = sfGFP of expression cassette
  source 1..690: mol_type = other DNA
actggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaa

SEQ ID NO: 6 - DNA, 51 bp, synthetic construct
  misc_feature 1..51: note = Terminator of expression cassette
  source 1..51: mol_type = other DNA
gacgaacaataaggcctccctaacggggggccttttttattgataacaaaa

SEQ ID NO: 7 - DNA, 2474 bp, synthetic construct
  misc_feature 1..2474: note = Xylose inducible sfGFP expression cassette
  source 1..2474: mol_type = other DNA
tcatgcatattctggaaaagaaagcatgtcggaaatcaccatagctatacatcccagcaccacagctctgtctttcagttttgaaacctctatcttaagattataaaactgggcgataaaggatctctggttaattacttccctcaatttttctaaaaacaactctccaaaaaatgatgccttattacctataattaccatttcaggattaaaaatatttacaagatttgccacgcctatccccatcttttcagcaacctcaagtatagccattctgcaaactctgcttccttcttttgctgcttgtattatccgagaaggagtgatttcatctacattttcccagcttatatacctatcctctactccttgttttacaagcttttttataacactcaaaagtgccctctcggatgcaaagttctcaagacagccaatattgccgcagctgcaaacatcgtcctgaaagttgatagtggtatgtccaacttcacctgcaaatcctgcagcacctctgaaaagtttgttgtcgataataattcctgcaccaagcccaattccaacactcaaataaatcaaatcactaacttttccccactcaccaaaccacttttcgcccagtgcgcctgcatttgcttcattgtcaatataaacagggaggttgaacttttgctgaacaattgacctcaaagggacattttgccatttcaaattgggagcaagaaggacagttccagactctttttctataatacctggaacaccaattccaatacctaaaatcccttttggagtttgtggcgccttttttactgatttttcaatcaggtcaaaaagaagccttaaaagtttttccttatcctcacctattttcatgttggcatattcttcaaaaataacttctcccacaaagtttgagagaataatatgaatatagtcaacacctaagtcaattccgatgattgaacctacatctttgtttacctgtaaaaggacaggtcttcttccgcctttggacttaccgtatcctttttctactacatacccttcttttatgagctcatcggtgaggtttgagactgttgctttatttaaatctacaagtttcgatatctttgtacgagatattatcttgttgtccaaaattgttttcaaaaccaaaagcttgtttatttgctttagtagcgtgtggttacccatagtttttttccccctttaatgataattaagatattgcttatacaataaattatattataaattaatctaaaaaaaaacaacatgaaataattactatttttagtgaaaatattacgttacaactcaaattgcgccgttttgcttatcctacgtcagttttttagcttattgtattaattttaccgggaggcaaaatataccgctgcctgaaacatttatagccaatacagttatcttgcctgtttcgtcggctgcaaaatgcattatttgacgggaattgcaacgtggtcggcatattaatgcaagttgaactgacaatctgccattattttttgcaaaataatctttgtcttgaaagaaggattttttgaaaaaatgaagaatatatattatagatattagtttgtttaataaacaaactaagtacacgtactggcatgtttaaaaaataaggtttagttaaaaaactgatttattatagaaggagagtgagttataaaatgcgtaaaggcgaagagctgttcactggtgtcgtccctattctggtggaactggatggtgatgtcaacggtcataagttttccgtgcgtggcgagggtgaaggtgacgcaactaatggtaaactgacgctgaagttcatctgtactactggtaaactgccggtaccttggccgactctggtaacgacgctgacttatggtgttcagtgctttgctcgttatccggaccatatgaagcagcatgacttcttcaagtccgccatgccggaaggctatgtgcaggaacgcacgatttcctttaaggatgacggcacgtacaaaacgcgtgcggaagtgaaatttgaaggcgataccctggtaaaccgcattgagctgaaaggcattgactttaaagaagacggcaatatcctgggccataagctggaatacaattttaacagccacaatgtttacatcaccgccgataaacaaaaaaatggcattaaagcgaattttaaaattcgccacaacgtggaggatggcagcgtgcagctggctgatcactaccagcaaaacactccaatcggtgatggtcctgttctgctgccagacaatcactatctgagcacgcaaagcgttctgtctaaagatccgaacgagaaacgcgatcatatggttctgctggagttcgtaaccgcagcgggcatcacgcatggtatggatgaactgtacaaatgatctagagacgaacaataaggcctccctaacggggggccttttttattgataacaaaa

SEQ ID NO: 8 - DNA, 200 bp, synthetic construct
  misc_feature 1..200: note = Caldicellulosiruptor bescii
  source 1..200: mol_type = other DNA
tgcaagttgaactgacaatctgccattattttttgcaaaataatctttgtcttgaaagaaggattttttgaaaaaatgaagaatatatattatagatattagtttgtttaataaacaaactaagtacacgtactggcatgtttaaaaaataaggtttagttaaaaaactgatttattatagaaggagagtgagttataaa

SEQ ID NO: 9 - protein, 438 aa, synthetic construct
  REGION 1..438: note = Caldicellulosiruptor bescii
MKYFKDIPEVKYEGPQSDNPFAFKYYNPDEIIDGKPLKDHLRFAIAYWHTFCATGSDPFGQPTIVRPWDKFSNRMDNAKARVEAAFEFFELLDVPFFCFHDRDIAPEGENLKESNKNLDEIVSLIKEYLKTSKTKVLWGTANLFSHPRYVHGAATSCNADVFAYAAAQVKKALEVTKELGGENYVFWGGREGYETLLNTDMGLELDNLARFLHMAVEYAKEIGFDGQFLIEPKPKEPTKHQYDFDSAHVYGFLKKYDLDKYFKLNIEVNHATLAGHDFHHELRFARINNMLGSIDANMGDLLLGWDTDQFPTDVRLTTLAMYEVIKAGGFDKGGLNFDAKVRRGSFELEDLVIGHIAGMDAFAKGFKIAYKLVKDGVFDKFIDERYKSYKEGIGAKIVSGEANFKMLEEYALSLDKIENKSGKQELLEMILNKYMFSE

SEQ ID NO: 10 - DNA, 200 bp, synthetic construct
  misc_feature 1..200: note = Caldicellulosiruptor acetigenus I77R1B
  source 1..200: mol_type = other DNA
tcaagttgaactaatggcgtgctattttctttggaaaaaatttttttgctttaaaagaaggattttttgaaaaaatgaagaatatatattataatgatgttagtttgtttaataaacaaactaattgtatacaagaattttgtttaaaacttagatgtgcttaaaaaatttttcattatagaaggagagtgagttataaa

SEQ ID NO: 11 - DNA, 198 bp, synthetic construct
  misc_feature 1..198: note = Caldicellulosiruptor danielii
  source 1..198: mol_type = other DNA
caagttgaactgacaatctgccattatttttgcaaaataatctttgttgtgaaagaaggattttttgaaaaaatgaagaatatatattatgatgatgttagtttgtttaataaacaaactaagtatacgtactgcatgttctaaattaaggtttagtttaaaaattacttttattatagaaggagagtgagttataaa

SEQ ID NO: 12 - DNA, 198 bp, synthetic construct
  misc_feature 1..198: note = Caldicellulosiruptor hydrothermalis
  source 1..198: mol_type = other DNA
aagttgaactgacaatctgccattatttttgcaaaataatctttgttgtgaaagaaggattttttgaaaaaatgaagaatatatattataatgatgttagtttgtttaataaacaaactaagtacgcgtactggcatgtttaaaaaataaggtttagttaaaaaaattatttattatagaaggagagtgagttataaa

SEQ ID NO: 13 - DNA, 198 bp, synthetic construct
  misc_feature 1..198: note = Caldicellulosiruptor kronotskyensis
  source 1..198: mol_type = other DNA
gcacaagtttaattgacaatatgcctttatttttaaaaacattctttgttttgaaagaaggattttttgaaaaagtggagaatatatattataataaacttagtttgtttaataaacaaactaagtatgtgtatggcatgtttaaaaataagatttagtcaaaaaattatatattatagaaggagagtgagttataaa

SEQ ID NO: 14 - DNA, 200 bp, synthetic construct
  misc_feature 1..200: note = Caldicellulosiruptor obsidiansis
  source 1..200: mol_type = other DNA
tcatacaagttaaattaatagtctgctacttttgcaaaacattctttgttttaaagaaggattttttgaaaaagtggagaatatatattataataaacttagtttgtttaataaacaaactaagtatgtttacaaacatttttaaaaaagtgggtttacgttaagaaattatttattatagaaggagagtgagttataaa

SEQ ID NO: 15 - DNA, 200 bp, synthetic construct
  misc_feature 1..200: note = Caldicellulosiruptor owensensis
  source 1..200: mol_type = other DNA
ttaaaatgcaagttgaactgacaatctgctattttttggcaaaaaatttttttgctttaaaagaaggattttttgaaaggatgaagaatatattataatgttagtttgtttaataaacaaactaattgtatgtaaggactttgtttaaaacttagatgtgcttaaaaattttttattatagaaggagagtgagttataaa

SEQ ID NO: 16 - protein, 399 aa, synthetic construct
  REGION 1..399: note = Caldicellulosiruptor bescii
MGNHTLLKQINKLLVLKTILDNKIISRTKISKLVDLNKATVSNLTDELIKEGYVVEKGYGKSKGGRRPVLLQVNKDVGSIIGIDLGVDYIHIILSNFVGEVIFEEYANMKIGEDKEKLLRLLFDLIEKSVKKAPQTPKGILGIGIGVPGIIEKESGTVLLAPNLKWQNVPLRSIVQQKFNLPVYIDNEANAGALGEKWFGEWGKVSDLIYLSVGIGLGAGIIIDNKLFRGAAGFAGEVGHTTINFQDDVCSCGNIGCLENFASERALLSVIKKLVKQGVEDRYISWENVDEITPSRIIQAAKEGSRVCRMAILEVAEKMGIGVANLVNIFNPEMVIIGNKASFFGELFLEKLREVINQRSFIAQFYNLKIEVSKLKDRAVVLGCIAMVISDMLSFPEYA

SEQ ID NO: 17 - protein, 399 aa, synthetic construct
  REGION 1..399: note = Caldicellulosiruptor saccharolyticus
MGNHTLLKQINKLLVLKTILDNKTISRAKISKLVDLNKATVSNLTDELIKEGFVIEKGYGHSKGGRRPVLLEVNKNVGLIIGIDLGVNYIHLILTNFIGEIVWEKSANIRLGETQERILEVLFELIGEAIKVAPQTQKGILGIGIGVPGIVEKTSGIVLIAPNLRWKDVPLKAMVEERFNLPVYIDNEANAGALGEKWFGNWGDVSHLVYVSVGIGIGAGIVIGDEIYRGAKGFAGEVGHTTIDFNDDVCSCGNVGCLENFASERALLSLIQKIVESGWEDEYINKKNVDKLDASYIIDSAKLGSKVALHAIKDIANKLGIGIANLVNIFNPDIVIIGNKASFMGDLFLEELRRIVFKRSFITQYHHVRVEISKLKDRACVLGCVAMVISDMLAFPDYV

SEQ ID NO: 18 - protein, 399 aa, synthetic construct
  REGION 1..399: note = Caldicellulosiruptor obsidiansis
MGNHALLKQINKLLILKTILDNKMISRAKISRLVDLNKATVSNLTDELIKEGYIVEKGYGKSKGGRRPVLLQVNKDVGSIIGIDLGVDYIHVILSNFIGEIIFEEYVNIKMEEPKEKLLNLLFDMIEKAIDKAPPTPKGILGIGIGVPGIVEKESGIVLIAPNLKWKNVHLKSIIEQRFNLPVYIDNEANAGALGEKWFGEWGKVSDLIYLSVGIGLGAGIIIDNKLFRGAAGFAGEVGHTTINFQDDVCSCGNIGCLENFASERALLSVIKKLVKEGAEDRYISCENVDEITPSQIIQAAMDGSRICRMAVLEVAEKMAIGIANLVNIFNPEIVIVGNKASFFGDLFLEKLREVVNQKSFIAQFYDLKIEVSKLKDRAVVLGCIAMVISDMLSFPEYV

SEQ ID NO: 19 - protein, 399 aa, synthetic construct
  REGION 1..399: note = Caldicellulosiruptor hydrothermalis
MGNHTLLKQINKLLVLKTILDNKIISRAKISRLVDLNKATVSNLTDELIKEGYVVEKGYGKSKGGRRPVLLQVNKDVGSIIGIDLGVDYIHIILSNFVGEVIFEEYANMKMGEDKEKLLDLLFDLIERAINRAPQTPKGILGIGIGVPGIVEKESGIVLIAPNLKWKNVHLKSIVQQRFNLPVYIDNEANAGALGEKWFGEWGKVTDLIYLSVGIGLGAGIIIDNKLFRGAAGFAGEVGHTTINFQDDVCSCGNIGCLENFASERALLSVIKKLVKEGAEDRYISCENVDEITPSQIIQAAKDGSRVCRMAVLEVAEKMAIGIANLVNIFNPEIVIIGNKVSFFGDLFLEKLREVVNQKSFIAQFYDLKIEVSKLKDRAVVLGCIAMVISDMLSFPEYT

SEQ ID NO: 20 - protein, 399 aa, synthetic construct
  REGION 1..399: note = Caldicellulosiruptor owensensis
MGNHALLKQINKLLILKTILDNKMISRAKISRLVDLNKATVSNLTDELIKEGYIVEKGYGKSKGGRRPVLLQVNKDVGSIIGIDLGVDYIHVILSNFIGEIIFEEYVNIKMKEPKEKLLNLLFDMIEKAIDKAPPTPKGILGIGIGVPGIVEKESGIVLIAPNLEWKNVHLKSIIEQKFNLPVYIDNEANAGALGEKWFGEWGKVSDLIYLSVGIGLGAGIIIDNKLFRGAAGFAGEVGHTTINFQDDVCSCGNIGCLENFASERALLSIIKKLVKEGAEDRYISCENVDEITPSQIIQAAMDGSRICRMAVLEVAEKMAIGIANLVNIFNPEIVIVGNKASFFGDLFLEKLREAVNQKSFIAQFYDLKIEVSKLKDRAVVLGCIAMVISDMLSFPEYA

SEQ ID NO: 21 - protein, 399 aa, synthetic construct
  REGION 1..399: note = Caldicellulosiruptor acetigenus I77R1B
MGNHSLIKQINKLLILKTILDKGVISRAKISRLVDLNKATVSNLTDELIKEGYVIEKGYGKSKGGRRPVLLQVNKDVGSIIGIDLGVDYIHIILSNFIGEIIFEEYVNLKIKEDNEKFLNIFFDLIQKAIDKAPETPKGILGIGIGVPGIVEKESGVVLVAPNLKWSNVPLKDIVQQRFNLPVYIDNEANAGALGEKWFGEWGKVTDLIYLSVGIGLGAGIIIDNKLFRGAAGFAGEVGHTTINFQDDVCSCGNIGCLENFASERALLSVIKKLVKEGAEDRYISCENIDEITPSQIIQAAKDGSRVCRMAVLEVAEKMGIGVANLVNIFNPEIVIIGNKASFFGDLFLEKLREVVNQRSFIAQFYNLKIEVSKLKDRAVVLGCIAMVISDMLSFPEYT

SEQ ID NO: 22 - protein, 399 aa, synthetic construct
  REGION 1..399: note = Caldicellulosiruptor kronotskyensis
MGNHTLLKQINKLLVLKTILDNKIISRAKISRLVDLNKATVSNLTDELIKEGYIVEKGYGKSKGGRRPVLLQVNKDVGSIIGIDLGVDYIHVILSNFVGEVIFEEYAEMKMGEDKDKLFDLLFDLIEKAIDRAPHTPKGILGIGIGVPGIVEKESGIVLIAPNLKWKNVHLKSIVQQRFNLPVYIDNEANAGALGEKWFGEWGKVSDLIYLSVGIGLGAGIIIDNKLFRGAAGFAGEVGHTTINFQDDVCSCGNIGCLENFASERALLSVIKKLVKQGVEDRYISCENVHEITPSRIIQAAKEGSRVCRMAILEVAEKMGIGVANLVNIFNPEMVIIGNKASFFGELFLEKLREVINQRSFIAQFYNLKIEVSKLKDRAVVLGCIAMVISDMLSFPEYA

SEQ ID NO: 23 - protein, 399 aa, synthetic construct
  REGION 1..399: note = Caldicellulosiruptor acetigenus 6A
MGNHSLIKQINKLLILKTILDKGVISRAKISRLVDLNKATVSNLTDELIKEGYVIEKGYGKSKGGRRPVLLQVNNDVGSIIGIDLGVDYIHIILSNFIGEIIFEEYVNLKIKEDNEKFLNIFFDLIQKAIDKAPETPKGILGIGIGVPGIVEKESGVVLVAPNLKWSNVPLKDIVKQRFNLPVYIDNEANAGALGEKWFGEWGKVTDLIYLSVGIGLGAGIIIDNKLFRGAAGFAGEVGHTTINFQDDVCSCGNIGCLENFASERALLSVIKKLVKEGAEDRYISCENIDEITPSQIIQAAKDGSRVCRMAVLEVAEKMGIGVANLVNIFNPEIVIIGNKASFFGDLFIEKLREVVNQRSFIAQFYNLKIEVSKLKDRAVVLGCIAMVISDMLSFPEYA

SEQ ID NO: 24 - protein, 399 aa, synthetic construct
  REGION 1..399: note = Caldicellulosiruptor changbaiensis
MGNHTLLKQINKLLVLKTILDNKTISRAKISRLVDLNKATVSNLTDELIREGFVIEKGYGHSKGGRRPVLLEVNKNVGLIIGIDLGVNYIHLVLTNFIGEIVWEKSANIRLGETQERILEVLFELISEAIKVAPQTQKGILGIGIGVPGIVEKTSGTVLIAPNLRWKDVPLKAMVEERFNLPVYIDNEANAGALGEKWFGDWGDVSHLVYVSVGIGIGAGIVIGDEIYRGAKGFAGEVGHTTIDFNDDVCSCGNVGCLENFASERALLSLIQKMVESSWEDEYINKKNVDKLDASYIIDSAKLGSKVALHAIKDIANKLGIGIANLVNIFNPDIVIIGNKASFMGDLFLEELRRIVFKRSFITQYHHVRVEVSRLKDRACVLGCVAMVISDMLSFPDYV

SEQ ID NO: 25 - protein, 399 aa, synthetic construct
  REGION 1..399: note = Caldicellulosiruptor naganoensis
MGNHTLLKQINKLLVLKTILDNKTISRAKISRLVDLNKATVSNLTDELIREGFVIEKGYGHSKGGRRPVLLEVNKNVGLIIGIDLGVNYIHLVLTNFIGEIVWEKSANIRLGETQERILEILFELISDAIKVAPQTQKGILGIGIGVPGIVEKTSGTVLIAPNLRWKDVPLKTMVEEKFNLPVYIDNEANAGALGEKWFGDWGDVSHLVYVSVGIGIGAGIVIGDEIYRGAKGFAGEVGHTTIDFNDDVCSCGNVGCLENFASERALLSLIQKIVESGWEDEYINKKNVDKLDASYIIDSAKLGSKVALHAIKDIANKLGIGIANLVNIFNPDIVIIGNKASFMGDLFLEELRRIVFKRSFITQYHRVRVEVSRLKDRACVLGCVAMVISDMLAFPDYV

SEQ ID NO: 26 - protein, 399 aa, synthetic construct
  REGION 1..399: note = Caldicellulosiruptor morganii
MGNHALIKQINKLLILKTILDHRTISRAKISRLVDLNKATVSNLTDELIKEGFVIEKGYGHSKGGRRPVLLEVNKNVGLIIGIDLGVNYIHLILTNFIGEIIWERNMAIKLGEKQEKILESLFMIIEEAIKAAPPTQKGILGIGIGVPGIVEKNSGTVLLAPNLKWQDVPLKRMVESRFGLPVYIDNEANAGALGEKWFGGWGDVSHLLYVSVGIGIGAGIVIGEEVYRGAKGFAGEVGHMTIDFNDDVCSCGNVGCLENFASERALLSLIKTLVESGVEDEYINRDTVEEMDAGYIIQSALQRSRVAMNAITDIANKLGIGIANLVNIFNPDIVVIGNKASFMGDLFLEELRRIVYKRAFITQYHHVRVEVSKLKDRACVLGCVAMVISDMLAFPDYV

SEQ ID NO: 27 - protein, 399 aa, synthetic construct
  REGION 1..399: note = Caldicellulosiruptor danielii
MGNHSLIKQINKLLILKTILDHKVISRAKISRLVDLNKATVSNLTDELIKEGYVIEKGYGKSKGGRRPVLLQVNKDVGSIIGIDLGVDYIHLILSNFVGEIIFEEYVNLRFGENKEKFLNTLFALIQKAIDKSPQTPKGILGIGIGVPGIVEKESGVVLIAPNLKWVNIPLKEILEDRFKLPVYIDNEANAGALGEKWFGEWGKVSDLIYLSVGIGLGAGIIIDNKLFRGAAGFAGEVGHTTINFQDDVCSCGNIGCLENFASERALLSVIKKLVKEGTEDRYVSCENVDEITPSQIIQAAKDGSRVCRMAVLEIAEKMGIGVANLVNIFNPEIVIIGNKASFFGDLFLEKLREVVNQKSFIAQFYNLKIEVSKLKDKAVVLGCIAMVISDMLSFPEYA

SEQ ID NO: 28 - protein, 399 aa, synthetic construct
  REGION 1..399: note = Caldicellulosiruptor diazotrophicus
MGNHTLLKQINKLLVLKTILDNKIISRAKISRLVDLNKATVSNLTDELIKEGYIVEKGYGKSKGGRRPVLLQVNKDVGSIIGIDLGVDYIHIILSNFVGEIIFEEYADIKMGENKEKLFELLFELIEKAIDKSPQTPKGILGIGIGVPGIVEKKSGIVLIAPNLKWKNVHLKSIVQQRFNLPVYIDNEANAGALGEKWFGEWGKVSDLIYLSVGIGLGAGIIIDNKLFRGAAGFAGEVGHTTINFQDDVCSCGNIGCLENFASERALLSVIKKLVKEGAEDRYISCENVDEITPFQIIQAAMDGSRVCRMAVLEVAEKMAIGIANLVNIFNPEIVIIGNKASFFGDLFLEKLREIVNQKSFIAQFYNLKIEVSKLKDRAVVLGCIAMVISDMLSFPEYA

SEQ ID NO: 29 - DNA, 18 bp, synthetic construct
  misc_feature 1..18: note = PCR Primer 10
  source 1..18: mol_type = other DNA
cggtttctttaccagact

SEQ ID NO: 30 - DNA, 20 bp, synthetic construct
  misc_feature 1..20: note = PCR Primer 11
  source 1..20: mol_type = other DNA
ggtattttacccttgtcgtt

SEQ ID NO: 31 - DNA, 22 bp, synthetic construct
  misc_feature 1..22: note = PCR Primer 13
  source 1..22: mol_type = other DNA
ctctgctactcgtttttcaatc

SEQ ID NO: 32 - DNA, 20 bp, synthetic construct
  misc_feature 1..20: note = PCR Primer 14
  source 1..20: mol_type = other DNA
gggaaggatttgaggttagg

SEQ ID NO: 33 - DNA, 23 bp, synthetic construct
  misc_feature 1..23: note = PCR Primer 49
  source 1..23: mol_type = other DNA
ggcaccaataactgccttaaaga

SEQ ID NO: 34 - DNA, 23 bp, synthetic construct
  misc_feature 1..23: note = PCR Primer 50
  source 1..23: mol_type = other DNA
cgatgataaacaaaggacggttc